(19) United States
(12) Patent Application Publication
(10) Pub. No.: US 2012/0321590 A1
(43) Pub. Date: Dec. 20, 2012

(54) BRIDGED POLYCYCLIC COMPOUNDS

(75) Inventors: Stephen E. Webber, San Diego, CA (US); Chinh V. Tran, San Diego, CA (US); Alan X. Xiang, Chula Vista, CA (US); Douglas E. Murphy, Fallbrook, CA (US)

(73) Assignee: Anadys Pharmaceuticals, Inc., San Diego, CA (US)

(21) Appl. No.: 13/439,959

(22) Filed: Apr. 5, 2012

Related U.S. Application Data
(60) Provisional application No. 61/472,286, filed on Apr. 6, 2011.

(51) Int. Cl. A61K 31/549; C07D 513/04; A61P 31/14; A61P 31/04; A61P 31/10; A61P 29/00; A61P 25/24; A61P 35/00; A61P 37/02; A61K 38/21; A61K 31/7056; A61K 38/19; A61K 31/7068; C12N 7/06; C07D 417/04

(52) U.S. Cl. 424/85.6; 544/13; 514/223.2; 544/10; 514/222.8; 424/85.7; 514/43; 514/49; 435/238

(57) ABSTRACT

The invention is directed to amides of bicyclic amine compounds and pharmaceutical compositions containing such compounds that are useful in treating infections by hepatitis C virus.

This application claims the benefit of U.S. Provisional Application No. 61/472,286, filed Apr. 6, 2011.

### FIELD OF THE INVENTION

The invention is directed to bridged polycyclic compounds and pharmaceutical compositions containing such compounds that are useful in treating infections by hepatitis C virus.

### BACKGROUND OF THE INVENTION

Hepatitis C is a major health problem world-wide. The World Health Organization estimates that 170 million people are chronic carriers of the hepatitis C virus (HCV), with 4 million carriers in the United States alone. In the United States, HCV infection accounts for 40% of chronic liver disease and HCV disease is the most common cause for liver transplantation. HCV infection leads to a chronic infection and about 70% of persons infected will develop chronic histological changes in the liver (chronic hepatitis) with a 10-40% risk of cirrhosis and an estimated 4% lifetime risk of hepatocellular carcinoma. The CDC estimates that each year in the United States there are 35,000 new cases of HCV infection and approximately ten thousand deaths attributed to HCV disease.

The current standard of care is a pegylated interferon/ribavirin combination at a cost of approximately $30,000/year. These drugs have difficult dosing problems and side-effects and do not achieve a sustained virological response in a significant number of diagnosed patients. Pegylated interferon treatment is associated with menacing flu-like symptoms, irritability, inability to concentrate, suicidal ideation, and leukocytopenia. Ribavirin is associated with hemolytic anemia and birth defects.

The overall response to this standard therapy is low; as approximately one third of patients do not respond. Of those who do respond, some relapse within six months of completing 6-12 months of therapy. As a consequence, the long-term response rate for all patients entering treatment is only about 50%. The relatively low response rate and the significant side-effects of current therapy anti-HCV drug treatments, coupled with the negative long term effects of chronic HCV infection, result in a continuing medical need for improved therapy. Antiviral pharmaceuticals to treat RNA virus diseases like HCV are few, and as described above are often associated with multiple adverse effects.

A number of publications have described NS5B inhibitors useful in the treatment of hepatitis C infection. See, e.g., International Publication Nos. WO 2010/0119481 and 2008/124450 (disclosing certain 5,6-dihydro-1H-pyridin-2-one compounds); U.S. Patent Application Publication No. US 2008/0031852 (describing [1,2-b]pyridazinone compounds); U.S. Patent Application Publication No. US 2006/0189602 (disclosing certain pyridazinones); U.S. Patent Application Publication No. US 2006/0252785 (disclosing selected heterocyclics); and International Publication Nos. WO 03/059356, WO 2002/098424, and WO 01/85172 (each describing a particular class of substituted thiadiazines).

While there are, in some cases, medicines available to reduce disease symptoms, there are few drugs to effectively inhibit replication of the underlying virus. The significance and prevalence of RNA virus diseases, including but not limited to chronic infection by the hepatitis C virus, and coupled with the limited availability and effectiveness of current antiviral pharmaceuticals, have created a compelling and continuing need for new pharmaceuticals to treat these diseases.

### SUMMARY OF THE INVENTION

The present invention describes novel bridged polycyclic compounds and pharmaceutically acceptable salts thereof, which are useful in treating or preventing a hepatitis C virus infection in a patient in need thereof comprising administering to the patient a therapeutically or prophylactically effective amount of a bridged polycyclic compound.

The invention relates to compounds of Formula I

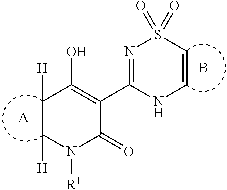

wherein
Ring A is selected from

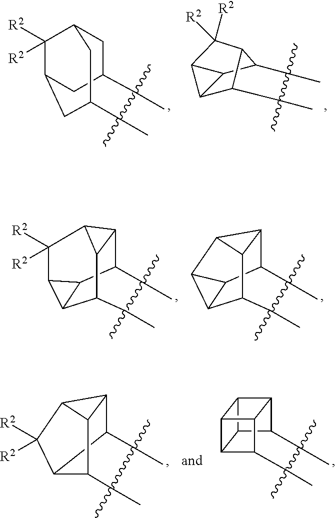

### Ring B is

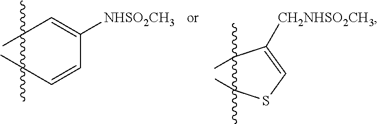

R¹ is alkyl or —C₁-C₆ alkylene(aryl),
R² is independently H, F, OH, OR³, —C₁-C₆ alkyl, C₃-C₈ cycloalkyl, —C₁-C₆ alkylene(C₃-C₈ cycloalkyl), —C₁-C₆ alkylene(aryl), —C₁-C₆ alkylene(heterocyclyl), aryl, or heterocyclyl, or both of the R² substituents are OCH₃, form an oxo, or form a ring comprised of —OCH₂CH₂O— or —SCH₂CH₂S—, and
R³ is a tert-butyl or CH₂Ph,
wherein the above alkyl, alkylene, cycloalkyl, aryl, and heterocyclyl moieties are optionally and independently substituted by 1-4 substituents selected from hydrogen, alkylamine, amino, aryl, cycloalkyl, heterocyclyl, azido, C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, C₂-C₆ alkynyl, carboxyl, cyano, halo, hydroxyl, or nitro,

or a pharmaceutically acceptable salt or tautomer thereof.

In one embodiment of the invention Ring A is

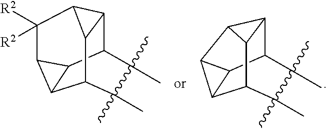

In one embodiment of the invention Ring B is

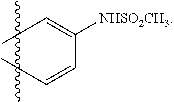

In one embodiment of the invention R¹ is —CH₂-aryl.

In one embodiment of the invention R² is H.

In one embodiment the invention is selected from the following compounds:
rac-N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide;
N-{3-[(4S,9R)-5-[(4-Fluorophenyl)methyl]-8-hydroxy-6-oxo-5-azahexacyclo[8.5.0.0^{2,15}.0^{3,12}.0^{11,13}]pentadec-7-en-7-yl]-1,1-dioxo-4H-1λ⁶,2,4-benzothiadiazin-7-yl}methanesulfonamide;
N-{3-[(4R,9S)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide;
rac-N-({3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide;
rac-N-{3-[(2S,7R)-3-[(4-Fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,2,4-benzothiadiazin-7-yl}methanesulfonamide;
rac-N-({3-[(2S,7R)-3-[(4-Fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,5,2,4-thieno[2,3-e][1λ⁶,2,4]thiadiazin-7-yl}methyl)methanesulfonamide;
rac-N-{3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide;
rac-N-({3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide;
rac-N-({3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,5,2,4-thieno[2,3-e][1λ⁶,2,4]thiadiazin-7-yl}methyl)methanesulfonamide;
rac-N-({3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide;
N-{3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide;
N-{3-[(1S,10R)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide;
rac-N-({3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,5,2,4-thieno[2,3-e][1λ⁶,2,4]thiadiazin-7-yl}methyl)methanesulfonamide;
rac-N-{3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,2,4-benzothiadiazin-7-yl}methanesulfonamide;
rac-N-({3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,5,2,4-thieno[2,3-e][1λ⁶,2,4]thiadiazin-7-yl}methyl)methanesulfonamide; and
rac-N-{3-[(4S,9R)-14,14-difluoro-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide.

In another embodiment the invention is selected from the following compounds:
rac-N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide;
N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide;
rac-N-({3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide; and
N-{3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide.

The invention is also directed to pharmaceutically acceptable salts and pharmaceutically acceptable solvates of the Formula I compounds of the invention. Advantageous methods of making Formula I compounds of the invention are also described.

In one aspect, the invention encompasses a method for treating or preventing hepatitis C virus infection in a mammal in need thereof, preferably in a human in need thereof, comprising administering to the patient a therapeutically or prophylactically effective amount of a Formula I compound of the invention.

In one aspect, the invention encompasses a method for treating or preventing hepatitis C virus infection by administering to a patient in need thereof a therapeutically or prophylactically effective amount of a Formula I compound of the invention that is an inhibitor of HCV NS5B polymerase.

In another aspect, the invention encompasses a method for treating or preventing hepatitis C virus infection by administering to a patient in need thereof a therapeutically or prophylactically effective amount of a Formula I compound that is an improved HCV genotype 1a NS5B inhibitor that also retains nanomolar activity against HCV genotype 1b NS5B polymerase.

In another aspect, the invention encompasses a method for treating or preventing hepatitis C virus infection in a patient in need thereof, comprising administering to the patient a therapeutically or prophylactically effective amount of a Formula I compound of the invention and a pharmaceutically acceptable excipient, carrier, or vehicle.

In another aspect, the invention encompasses a method for treating or preventing hepatitis C virus infection in a patient in need thereof, comprising administering to the patient a therapeutically or prophylactically effective amount of a Formula I compound of the invention and one or more additional therapeutic agents, preferably an additional antiviral agent or an immunomodulatory agent.

### DETAILED DESCRIPTION OF THE INVENTION

Where the following terms are used in this specification, they are used as defined below:

The terms “comprising,” “having” and “including” are used herein in their open, non-limiting sense.

The term “Me” means methyl, “Et” means ethyl, and “Ac” means acetyl.

The term “alkyl”, as used herein, unless otherwise indicated, includes C₁-C₆ saturated monovalent hydrocarbon radicals having straight, branched, or cyclic moieties (including fused and bridged bicyclic and spirocyclic moieties), or a combination of the foregoing moieties. For an alkyl group to have cyclic moieties, the group must have at least three carbon atoms.

The term “alkylene”, as used herein, unless otherwise indicated, includes a divalent radical derived from alkyl, as exemplified by —CH₂CH₂CH₂CH₂—.

The term “alkoxy”, as used herein, unless otherwise indicated, includes O-alkyl groups wherein alkyl is as defined above.

The term “cycloalkyl”, as used herein, unless otherwise indicated refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary cycloalkyls include monocyclic rings having from 3-7, preferably 3-6, carbon atoms, such as cyclopropyl, cyclobutyl, cyclopentyl, cyclohexyl, cycloheptyl and the like. Illustrative examples of cycloalkyl are derived from, but not limited to, the following:

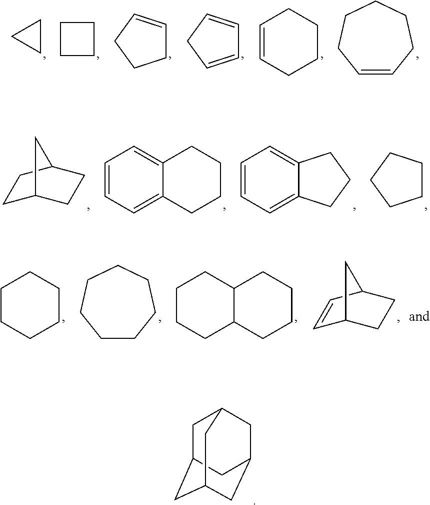

The term “aryl”, as used herein, unless otherwise indicated, includes an organic radical derived from an aromatic hydrocarbon by removal of one hydrogen, and has from 6-14 carbon atoms in its ring system, such as phenyl or naphthyl.

The term “heterocyclic” or “heterocyclyl”, as used herein, unless otherwise indicated, includes aromatic (e.g., heteroaryls) and non-aromatic heterocyclic groups containing one to four heteroatoms each selected from O, S and N, wherein each heterocyclic group has from 4-10 atoms in its ring system, and with the proviso that the ring of said group does not contain two adjacent O atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other illustrative examples of 4-10 membered heterocyclic are derived from, but not limited to, the following:

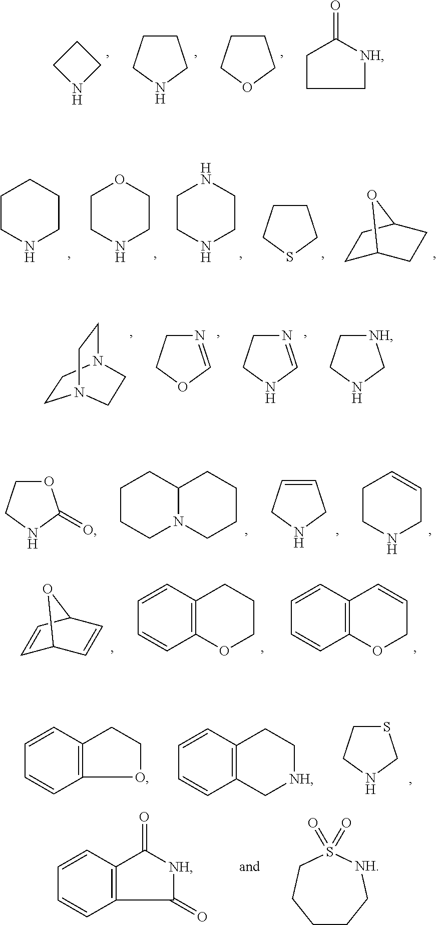

Unless defined otherwise each “alkyl” is optionally and independently substituted by 1-3 substituents selected from amino, cyano, halo, hydroxy, nitro, C₁-C₆ alkylamine, C₁-C₆ dialkylamine, C₁-C₆ alkyl, C₁-C₆ alkoxy, C₁-C₆ alkenyl, and C₁-C₆ hydroxyalkyl, wherein each alkyl is optionally substituted by one or more halo substituents, e.g., CF₃.

The term “immunomodulator” refers to natural or synthetic products capable of modifying the normal or aberrant immune system through stimulation or suppression.

The term “preventing” refers to the ability of a compound or composition of the invention to prevent a disease identified herein in patients diagnosed as having the disease or who are at risk of developing such disease. The term also encompasses preventing further progression of the disease in patients who are already suffering from or have symptoms of such disease.

The term “patient” or “subject” means an animal (e.g., cow, horse, sheep, pig, chicken, turkey, quail, cat, dog, mouse, rat, rabbit, guinea pig, etc.) or a mammal, including chimeric and transgenic animals and mammals. In the treatment or prevention of HCV infection, the term “patient” or “subject” preferably means a monkey or a human, most preferably a human. In a specific embodiment the patient or subject is infected by or exposed to the hepatitis C virus. In certain embodiments, the patient is a human infant (age 0-2), child (age 2-17), adolescent (age 12-17), adult (age 18 and up) or geriatric (age 70 and up) patient. In addition, the patient includes immunocompromised patients such as HIV positive patients, cancer patients, patients undergoing immunotherapy or chemotherapy. In a particular embodiment, the patient is a healthy individual, i.e., not displaying symptoms of other viral infections.

The term a “therapeutically effective amount” refers to an amount of the compound of the invention sufficient to provide a benefit in the treatment or prevention of viral disease, to delay or minimize symptoms associated with viral infection or viral-induced disease, or to cure or ameliorate the disease or infection or cause thereof. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent.

The term a “prophylactically effective amount” refers to an amount of a compound of the invention or other active ingredient sufficient to result in the prevention of infection, recurrence or spread of viral infection. A prophylactically effective amount may refer to an amount sufficient to prevent initial infection or the recurrence or spread of the infection or a disease associated with the infection. Used in connection with an amount of a compound of the invention, the term preferably encompasses a non-toxic amount that improves overall prophylaxis or enhances the prophylactic efficacy of or synergies with another prophylactic or therapeutic agent.

The term “in combination” refers to the use of more than one prophylactic and/or therapeutic agents simultaneously or sequentially and in a manner that their respective effects are additive or synergistic.

The term “treating” refers to:

(i) preventing a disease, disorder, or condition from occurring in an animal that may be predisposed to the disease, disorder and/or condition, but has not yet been diagnosed as having it;

(ii) inhibiting the disease, disorder, or condition, i.e., arresting its development; and

(iii) relieving the disease, disorder, or condition, i.e., causing regression of the disease, disorder, and/or condition.

The terms “R” and “S” indicate the specific stereochemical configuration of a substituent at an asymmetric carbon atom in a chemical structure as drawn.

The term “rac” indicates that a compound is a racemate, which is defined as an equimolar mixture of a pair of enantiomers. A “rac” compound does not exhibit optical activity. The chemical name or formula of a racemate is distinguished from those of the enantiomers by the prefix (±)- or rac- (or racem-) or by the symbols RS and SR.

The terms “cis” and “trans” are descriptors which show the relationship between two ligands attached to separate atoms that are connected by a double bond or are contained in a ring. The two ligands are said to be located cis to each other if they lie on the same side of a plane. If they are on opposite sides, their relative position is described as trans. The appropriate reference plane of a double bond is perpendicular to that of the relevant σ-bonds and passes through the double bond. For a ring it is the mean plane of the ring(s).

The compounds of the invention may exhibit the phenomenon of tautomerism. While Formula I cannot expressly depict all possible tautomeric forms, it is to be understood that Formula I is intended to represent any tautomeric form of the depicted compound and is not to be limited merely to a specific compound form depicted by the formula drawings. For illustration, and in no way limiting the range of tautomers, the compounds of Formula I may exist as the following:

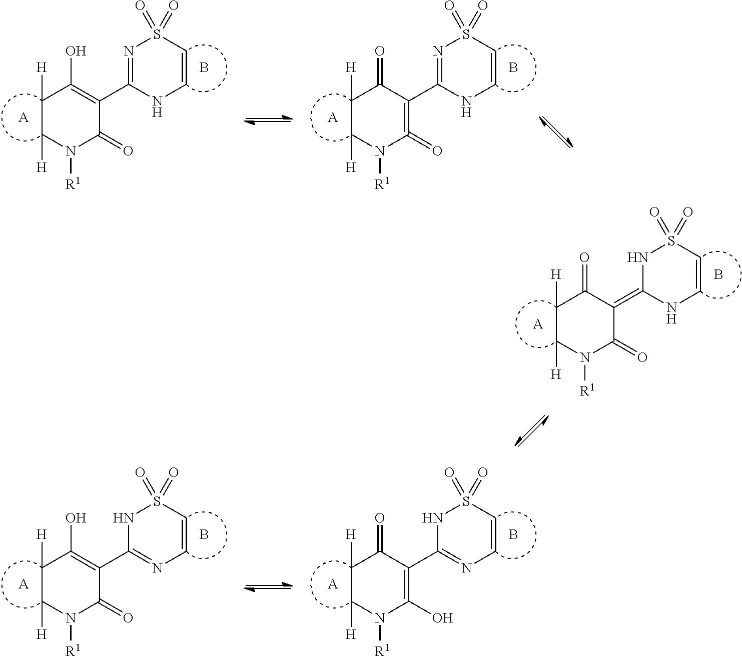

Some of the inventive Formula I compounds may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% free of other enantiomers or diastereomers of the compounds, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, Formula I compounds of the invention are intended to cover solvated as well as unsolvated forms of the identified structures. For example, the invention includes compounds of the indicated structure in both hydrated and non-hydrated forms. Other examples of solvates include the structures in combination with isopropanol, ethanol, methanol, DMSO, ethyl acetate, pentyl acetate, acetic acid, or ethanolamine.

In addition to compounds of the invention, the invention includes pharmaceutically acceptable prodrugs, pharmaceutically active metabolites, and pharmaceutically acceptable salts of such compounds and metabolites.

“A pharmaceutically acceptable prodrug” is a compound that may be converted under physiological conditions or by solvolysis to the specified compound or to a pharmaceutically acceptable salt of such compound prior to exhibiting its pharmacological effect (s). Typically, the prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the compounds of the invention using methods known in the art, such as those described by Burger's Medicinal Chemistry and Drug Chemistry, 1, 172-178, 949-982 (1995). See also Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

“A pharmaceutically active metabolite” is intended to mean a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect. For example, anticancer drugs of the anti-metabolite class must be converted to their active forms after they have been transported into a cancer cell.

Since most drugs undergo metabolic transformation of some kind, the biochemical reactions that play a role in drug metabolism may be numerous and diverse. The main site of drug metabolism is the liver, although other tissues may also participate.

A feature characteristic of many of these transformations is that the metabolic products, or “metabolites,” are more polar than the parent drugs, although a polar drug does sometime yield a less polar product. Substances with high lipid/water partition coefficients, which pass easily across membranes, also diffuse back readily from tubular urine through the renal tubular cells into the plasma. Thus, such substances tend to have a low renal clearance and a long persistence in the body. If a drug is metabolized to a more polar compound, one with a lower partition coefficient, its tubular reabsorption will be greatly reduced. Moreover, the specific secretory mechanisms for anions and cations in the proximal renal tubules and in the parenchymal liver cells operate upon highly polar substances.

As a specific example, phenacetin (acetophenetidin) and acetanilide are both mild analgesic and antipyretic agents, but are transformed within the body to a more polar and more effective metabolite, p-hydroxyacetanilid (acetaminophen), which is widely used today. When a dose of acetanilide is given to a person, the successive metabolites peak and decay in the plasma sequentially. During the first hour, acetanilide is the principal plasma component. In the second hour, as the acetanilide level falls, the metabolite acetaminophen concentration reaches a peak. Finally, after a few hours, the principal plasma component is a further metabolite that is inert and can be excreted from the body. Thus, the plasma concentrations of one or more metabolites, as well as the drug itself, can be pharmacologically important.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal, co-crystal, or polymorphic forms, all of which are intended to be within the scope of the present invention and specified formulas.

### Methods of Treatment and Prevention of Hepatitis C Viral Infections

The present invention provides methods for treating or preventing a hepatitis C virus infection in a patient in need thereof.

The present invention further provides methods for introducing a therapeutically effective amount of the Formula I compounds of the invention or combination of such compounds into the blood stream of a patient in the treatment and/or prevention of hepatitis C viral infections.

The magnitude of a prophylactic or therapeutic dose of a compound of the invention or a pharmaceutically acceptable salt, solvate, or hydrate, thereof in the acute or chronic treatment or prevention of an infection will vary, however, with the nature and severity of the infection, and the route by which the active ingredient is administered. The dose, and in some cases the dose frequency, will also vary according to the infection to be treated, the age, body weight, and response of the individual patient. Suitable dosing regimens can be readily selected by those skilled in the art with due consideration of such factors.

The methods of the present invention are particularly well suited for human patients. In particular, the methods and doses of the present invention can be useful for immunocompromised patients including, but not limited to cancer patients, HIV infected patients, and patients with an immunodegenerative disease. Furthermore, the methods can be useful for immunocompromised patients currently in a state of remission. The methods and doses of the present invention are also useful for patients undergoing other antiviral treatments. The prevention methods of the present invention are particularly useful for patients at risk of viral infection. These patients include, but are not limited to health care workers, e.g., doctors, nurses, hospice care givers; military personnel; teachers; childcare workers; patients traveling to, or living in, foreign locales, in particular third world locales including social aid workers, missionaries, and foreign diplomats. Finally, the methods and compositions include the treatment of refractory patients or patients resistant to treatment such as resistance to reverse transcriptase inhibitors, protease inhibitors, etc.

### Doses

Toxicity and efficacy of the compounds of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage of the compounds for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture; alternatively, the dose of the compound of the invention may be formulated in animal models to achieve a circulating plasma concentration range of the compound that corresponds to the concentration required to achieve a fixed magnitude of response. Such information can be used to more accurately determine useful doses in humans. Levels in plasma may be measured, for example, by high performance liquid chromatography.

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which cells that are responsive to the effects of the compounds of the invention are exposed to the ligand and the magnitude of response is measured by an appropriate technique. The assessment of the compound of the invention is then evaluated with respect to the potency of the compound of the invention, and the degree of conversion of the compound of the invention prodrug. Compounds for use in methods of the invention can be tested in suitable animal model systems prior to testing in humans, including but not limited to in rats, mice, chicken, cows, monkeys, rabbits, hamsters, etc. The compounds can then be used in the appropriate clinical trials.

The magnitude of a prophylactic or therapeutic dose of a prodrug of a compound of the invention or a pharmaceutically acceptable salt, solvate, or hydrate thereof in the acute or chronic treatment or prevention of an infection or condition will vary with the nature and severity of the infection, and the route by which the active ingredient is administered. The dose, and perhaps the dose frequency, will also vary according to the infection to be treated, the age, body weight, and response of the individual patient. Suitable dosing regimens can be readily selected by those skilled in the art with due consideration of such factors. In one embodiment, the dose administered depends upon the specific compound to be used, and the weight and condition of the patient. Also, the dose may differ for various particular compounds of the invention; suitable doses can be predicted on the basis of the aforementioned in vitro measurements and on the basis of animal studies, such that smaller doses will be suitable for those compounds of the invention that show effectiveness at lower concentrations than other compounds of the invention when measured in the systems described or referenced herein. In general, the dose per day is in the range of from about 0.001 to 100 mg/kg, preferably about 1 to 25 mg/kg, more preferably about 5 to 15 mg/kg. For treatment of humans infected by hepatitis C viruses, about 0.1 mg to about 15 g per day is administered in about one to four divisions a day, preferably 100 mg to 12 g per day, more preferably from 100 mg to 8000 mg per day.

Additionally, the recommended daily dose ran can be administered in cycles as single agents or in combination with other therapeutic agents. In one embodiment, the daily dose is administered in a single dose or in equally divided doses. In a related embodiment, the recommended daily dose can be administered once time per week, two times per week, three times per week, four times per week or five times per week.

In one embodiment, the Formula I compounds of the invention are administered to provide systemic distribution of the compound within the patient. In a related embodiment, the compounds of the invention are administered to produce a systemic effect in the body.

In another embodiment the Formula I compounds of the invention are administered via oral, mucosal (including sublingual, buccal, rectal, nasal, or vaginal), parenteral (including subcutaneous, intramuscular, bolus injection, intraarterial, or intravenous), transdermal, or topical administration. In a specific embodiment the compounds of the invention are administered via mucosal (including sublingual, buccal, rectal, nasal, or vaginal), parenteral (including subcutaneous, intramuscular, bolus injection, intraarterial, or intravenous), transdermal, or topical administration. In a further specific embodiment, the compounds of the invention are administered via oral administration. In a further specific embodiment, the compounds of the invention are not administered via oral administration.

Different therapeutically effective amounts may be applicable for different infections, as will be readily known by those of ordinary skill in the art. Similarly, amounts sufficient to treat or prevent such infections, but insufficient to cause, or sufficient to reduce, adverse effects associated with conventional therapies are also encompassed by the above described dosage amounts and dose frequency schedules.

### Combination Therapy

Specific methods of the invention further comprise the administration of an additional therapeutic agent (i.e., a therapeutic agent other than a compound of the invention). In certain embodiments of the present invention, the compounds of the invention can be used in combination with at least one other therapeutic agent. Therapeutic agents include, but are not limited to antibiotics, antiemetic agents, antidepressants, and antifungal agents, anti-inflammatory agents, antiviral agents, anticancer agents, immunomodulatory agents, α-interferons, β-interferons, ribavirin, alkylating agents, hormones, cytokines, or toll-like receptor modulators. In one embodiment the invention encompasses the administration of an additional therapeutic agent that is HCV specific or demonstrates anti-HCV activity.

The Formula I compounds of the invention can be administered or formulated in combination with antibiotics. For example, they can be formulated with a macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The Formula I compounds of the invention can also be administered or formulated in combination with an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine, tropisetron, and mixtures thereof.

The Formula I compounds of the invention can be administered or formulated in combination with an antidepressant. Suitable antidepressants include, but are not limited to, binedaline, caroxazone, citalopram, dimethazan, fencamine, indalpine, indeloxazine hydrochloride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

The Formula I compounds of the invention can be administered or formulated in combination with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofulvin.

The Formula I compounds of the invention can be administered or formulated in combination with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

The Formula I compounds of the invention can be administered or formulated in combination with another antiviral agent. Useful antiviral agents include, but are not limited to, protease inhibitors, nucleoside reverse transcriptase inhibitors, non-nucleoside reverse transcriptase inhibitors and nucleoside analogs. The antiviral agents include but are not limited to zidovudine, acyclovir, gangcyclovir, vidarabine, idoxuridine, trifluridine, levovirin, viramidine, ribavirin, and taribavirin, as well as foscarnet, amantadine, rimantadine, saquinavir, indinavir, amprenavir, lopinavir, ritonavir, the α-interferons, β-interferons, adefovir, clevadine, entecavir, pleconaril, BMS-824393, and GI-5005.

The Formula I compounds of the invention can be administered or formulated in combination with an immunomodulatory agent. Immunomodulatory agents include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boehringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), anti-B7 antibodies (e.g., IDEC-114 (IDEC)), CTLA4-immunoglobulin, and toll-like receptor (TLR) modulators (e.g., ANA773, IMO-2125, PF-04878691, SD-101, GS-9620). Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies).

The Formula I compounds of the invention can be administered or formulated in combination with an agent which inhibits viral enzymes, including but not limited to inhibitors of HCV protease, such as VX-500, VBY-376, BMS-650032, MK-7009 (vaniprevir), TMC-435350, BI-201335, SCH-503034 (boceprevir), ITMN-191 (danoprevir), VX-950 (telaprevir), SCH900518 (narlaprevir), VX-813, VX-985, PHX1766, ABT-450, ACH-1625, ACH-1095, IDX136, IDX316, GS-9451, GS-9256, IDX-320, Merck-5172, and ITMN-5489; inhibitors of NS5B polymerase such as GS-9190, MK-3281, VCH-759 (VX-759), VCH-916, ABT-333, BMS-791325, PF-00868554 (filibuvir), IDX-184, IDX-375, R7128, RG7348, PSI-938, PSI-6130, PSI-7977, R1626, PSI-7851, VCH-222 (VX-222), ABT-072, INX-189, BI207127, TMC-647055, and ANA598; and inhibitors of the NS5A protein, such as BMS-790052, BMS-824393, A-831, GS-5885, Presidio-461, AZD-7295, and AZD2836.

The Formula I compounds of the invention can be administered or formulated in combination with an agent which inhibits HCV polymerase such as those described in Wu, Curr Drug Targets Infect Disord. 2003, 3 (3), 207-19 or in combination with compounds that inhibit the helicase function of the virus such as those described in Bretner M, et al. Nucleosides Nucleotides Nucleic Acids. 2003, 22 (5-8), 1531, or with inhibitors of other HCV specific targets such as those described in Zhang X., IDrugs 2002, 5 (2), 154-8.

The Formula I compounds of the invention can be administered or formulated in combination with an agent which inhibits viral replication.

The Formula I compounds of the invention can be administered or formulated in combination with an agent which inhibits cyclophilins. Examples of cyclophilin inhibitors include, but are not limited to, Debio-025, NIM-811, and SCY-635.

The Formula I compounds of the invention can be administered or formulated in combination with cytokines. Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-α, and IFN-γ).

The Formula I compounds of the invention can be administered or formulated in combination with hormones. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

The Formula I compounds of the invention can be administered or formulated in combination with β-interferons which include, but are not limited to, interferon β-1a, interferon β-1b.

The Formula I compounds of the invention can be administered or formulated in combination with α-interferons which include, but are not limited to, interferon α-1, interferon α-2a (roferon), interferon α-2b, intron, Peg-Intron, Pegasys, consensus interferon (infergen) and albuferon. The compounds of the invention can also be administered or formulated in combination with interferons such as BLX-883 (Locteron), ITCA-638, Omega interferon, and PEG-Interferon lambda.

The Formula I compounds of the invention can be administered or formulated in combination with an absorption enhancer, particularly those which target the lymphatic system, including, but not limited to sodium glycocholate; sodium caprate; N-lauryl-β-D-maltopyranoside; EDTA; mixed micelle; and those reported in Muranishi Crit. Rev. Ther. Drug Carrier Syst., 7, 1-33, which is hereby incorporated by reference in its entirety. Other known absorption enhancers can also be used. Thus, the invention also encompasses a pharmaceutical composition comprising one or more compounds of the invention and one or more absorption enhancers.

The Formula I compounds of the invention can be administered or formulated in combination with a cytochrome P450 monooxygenase inhibitor, such as, but not limited to, ritonavir or a pharmaceutically acceptable salt, ester, and prodrug thereof to improve the pharmacokinetics (e.g., increased half-life, increased time to peak plasma concentration, increased blood levels) of a compound of the invention that is metabolized by cytochrome P450 monooxygenase. Thus, the invention also encompasses a pharmaceutical composition comprising compounds of the invention and one or more cytochrome P450 monooxygenase inhibitors.

The Formula I compounds of the invention can be administered in combination with food to enhance absorption of the compounds of the invention in the gastrointestinal tract and to increase the bioavailability of the compounds of the invention.

The Formula I compounds of the invention can be administered or formulated in combination with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The Formula I compounds of the invention and the other therapeutics agent can act additively or, more preferably, synergistically. In one embodiment, a composition comprising a compound of the invention is administered concurrently with the administration of another therapeutic agent, which can be part of the same composition or in a different composition from that comprising the compounds of the invention. In another embodiment, a compound of the invention is administered prior to or subsequent to administration of another therapeutic agent. In a separate embodiment, a compound of the invention is administered to a patient who has not previously undergone or is not currently undergoing treatment with another therapeutic agent, particularly an antiviral agent.

In one embodiment, the methods of the invention comprise the administration of one or more compounds of the invention without an additional therapeutic agent.

### Pharmaceutical Compositions and Dosage Forms

Pharmaceutical compositions and single unit dosage forms comprising a compound of the invention, or a pharmaceutically acceptable salt, or hydrate thereof, are also encompassed by the invention. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, or topical administration. Pharmaceutical compositions and dosage forms of the invention typically also comprise one or more pharmaceutically acceptable excipients. Sterile dosage forms are also contemplated.

In an alternative embodiment, pharmaceutical composition encompassed by this embodiment includes a compound of the invention, or a pharmaceutically acceptable salt, or hydrate thereof, and at least one additional therapeutic agent. Examples of additional therapeutic agents include, but are not limited to, those listed above.

The composition, shape, and type of dosage forms of the invention will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990). Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise compounds of the invention, or a pharmaceutically acceptable salt or hydrate thereof comprise 0.1 mg to 1500 mg per unit to provide doses of about 0.01 to 200 mg/kg per day.

### Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry and/or lyophylized products ready to be dissolved or suspended in a pharmaceutically acceptable vehicle for injection (reconstitutable powders), suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### Transdermal Dosage Forms

Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Topical Dosage Forms

Topical dosage forms of the invention include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985).

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

### Mucosal Dosage Forms

Mucosal dosage forms of the invention include, but are not limited to, ophthalmic solutions, sprays and aerosols, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. In one embodiment, the aerosol comprises a carrier. In another embodiment, the aerosol is carrier free.

The compounds of the invention may also be administered directly to the lung by inhalation. For administration by inhalation, a compound of the invention can be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas can be used to deliver a compound of the invention directly to the lung. MDI devices are available from a number of suppliers such as 3M Corporation, Aventis, Boehringer Ingelheim, Forest Laboratories, Glaxo-Wellcome, Schering Plough and Vectura.

Alternatively, a Dry Powder Inhaler (DPI) device can be used to administer a compound of the invention to the lung (see, e.g., Raleigh et al., Proc. Amer. Assoc. Cancer Research Annual Meeting, 1999, 40, 397, which is herein incorporated by reference). DPI devices typically use a mechanism such as a burst of gas to create a cloud of dry powder inside a container, which can then be inhaled by the patient. DPI devices are also well known in the art and can be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator can be formulated containing a powder mix of the compound and a suitable powder base such as lactose or starch for these systems.

Another type of device that can be used to deliver a compound of the invention to the lung is a liquid spray device supplied, for example, by Aradigm Corporation. Liquid spray systems use extremely small nozzle holes to aerosolize liquid drug formulations that can then be directly inhaled into the lung.

In one embodiment, a nebulizer device is used to deliver a compound of the invention to the lung. Nebulizers create aerosols from liquid drug formulations by using, for example, ultrasonic energy to form fine particles that can be readily inhaled (See e.g., Verschoyle et al., British J. Cancer, 1999, 80, Suppl 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974, which are herein incorporated by reference), Aventis and Batelle Pulmonary Therapeutics.

In one embodiment, an electrohydrodynamic (“EHD”) aerosol device is used to deliver compounds of the invention to the lung. EHD aerosol devices use electrical energy to aerosolize liquid drug solutions or suspensions (see, e.g., Noakes et al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807, which are herein incorporated by reference). The electrochemical properties of the compounds of the invention formulation may be important parameters to optimize when delivering this drug to the lung with an EHD aerosol device and such optimization is routinely performed by one of skill in the art. EHD aerosol devices may more efficiently delivery drugs to the lung than existing pulmonary delivery technologies. Other methods of intra-pulmonary delivery of compounds of the invention will be known to the skilled artisan and are within the scope of the invention.

Liquid drug formulations suitable for use with nebulizers and liquid spray devices and EHD aerosol devices will typically include a compound of the invention with a pharmaceutically acceptable carrier. Preferably, the pharmaceutically acceptable carrier is a liquid such as alcohol, water, polyethylene glycol or a perfluorocarbon. Optionally, another material may be added to alter the aerosol properties of the solution or suspension of the compound of the invention. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. Nos. 5,112,598; Biesalski, 5,556,611, which are herein incorporated by reference) A compound of the invention can also be formulated in rectal or vaginal compositions such as suppositories or retention enemas, e.g., containing conventional suppository bases such as cocoa butter or other glycerides.

In addition to the formulations described previously, a compound of the invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems can be employed. Liposomes, emulsions, self-emulsifying (SEDDS), and self micro-emulsifying systems (SMEDDS) are well known examples of delivery vehicles that can be used to deliver compositions of the invention. Such systems can also contain fatty acids, bile salts and mixtures of mono-, di- and triglycerides to ameliorate potential food effects. Other functional lipid excipients include esters of glycerol, PEG-esters, propylene glycol esters and polyglycerol esters. Certain organic solvents such as dimethylsulfoxide can also be employed, although usually at the cost of greater toxicity. A compound of the invention can also be delivered in a controlled release system. In one embodiment, a pump can be used (Sefton, CRC Crit. Ref Biomed Eng., 1987, 14, 201; Buchwald et al., Surgery, 1980, 88, 507; Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem., 1983, 23, 61; see also Levy et al., Science, 1985, 228, 190; During et al., Ann. Neurol., 1989, 25, 351; Howard et al., J. Neurosurg., 71, 105 (1989). In yet another embodiment, a controlled-release system can be placed in proximity of the target of the compounds of the invention, e.g., the lung, thus requiring only a fraction of the systemic dose (see, e.g., Goodson, in Medical Applications of Controlled Release, supra, vol. 2, pp. 115 (1984)). Other controlled-release system can be used (see, e.g., Langer, Science, 1990, 249, 1527).

Suitable excipients (e.g., carriers and diluents) and other materials that can be used to provide mucosal dosage forms encompassed by this invention are well known to those skilled in the pharmaceutical arts, and depend on the particular site or method which a given pharmaceutical composition or dosage form will be administered. With that fact in mind, typical excipients include, but are not limited to, water, ethanol, ethylene glycol, propylene glycol, butane-1,3-diol, isopropyl myristate, isopropyl palmitate, mineral oil, and mixtures thereof, which are non-toxic and pharmaceutically acceptable. Examples of such additional ingredients are well known in the art. See, e.g., Remington's Pharmaceutical Sciences, 18th eds., Mack Publishing, Easton Pa. (1990).

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Kits

The invention provides a pharmaceutical pack or kit comprising one or more containers comprising a compound of the invention useful for the treatment or prevention of a Hepatitis C virus infection. In other embodiments, the invention provides a pharmaceutical pack or kit comprising one or more containers comprising a compound of the invention useful for the treatment or prevention of a Hepatitis C virus infection and one or more containers comprising an additional therapeutic agent, including but not limited to those listed above, in particular an antiviral agent, an interferon, an agent which inhibits viral enzymes, or an agent which inhibits viral replication, preferably the additional therapeutic agent is HCV specific or demonstrates anti-HCV activity.

The invention also provides a pharmaceutical pack or kit comprising one or more containers comprising one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The inventive agents may be prepared using the reaction routes and synthesis schemes as described below, employing the general techniques known in the art using starting materials that are readily available. The synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

### Preparation of Compounds

In the synthetic schemes described below, unless otherwise indicated all temperatures are set forth in degrees Celsius and all parts and percentages are by weight. Reagents were purchased from commercial suppliers such as Aldrich Chemical Company or Alfa Aesar. and were used without further purification unless otherwise indicated. All solvents were purchased from commercial suppliers such as Aldrich, EMD Chemicals or Fisher and used as received.

The reactions set forth below were done generally under a positive pressure of argon or nitrogen at an ambient temperature (unless otherwise stated) in anhydrous solvents, and the reaction flasks were fitted with rubber septa for the introduction of substrates and reagents via syringe. Glassware was oven dried and/or heat dried.

The reactions were assayed by TLC and/or analyzed by LC-MS or HPLC and terminated as judged by the consumption of starting material. Analytical thin layer chromatography (TLC) was performed on glass-plates precoated with silica gel 60 F₂₅₄ 0.25 mm plates (EMD Chemicals), and visualized with UV light (254 nm) and/or iodine on silica gel and/or heating with TLC stains such as ethanolic phosphomolybdic acid, ninhydrin solution, potassium permanganate solution or ceric sulfate solution. Preparative thin layer chromatography (prepTLC) was performed on glass-plates precoated with silica gel 60 F₂₅₄ 0.5 mm plates (20×20 cm, from Thomson Instrument Company) and visualized with UV light (254 nm).

Work-ups were typically done by doubling the reaction volume with the reaction solvent or extraction solvent and then washing with the indicated aqueous solutions using 25% by volume of the extraction volume unless otherwise indicated. Product solutions were dried over anhydrous sodium sulfate and/or magnesium sulfate prior to filtration and evaporation of the solvents under reduced pressure on a rotary evaporator and noted as solvents removed in vacuo. Column chromatography was completed under positive pressure using Merck silica gel 60, 230-400 mesh or 50-200 mesh neutral alumina, Teledyne Isco flash-chromatography using prepacked RediSep silica gel columns, or Analogix flash column chromatography using prepacked SuperFlash silica gel columns. Hydrogenolysis was done at the pressure indicated in the examples or at ambient pressure.

¹H-NMR spectra and ¹³C-NMR were recorded on a Varian Mercury-VX400 instrument operating at 400 MHz. NMR spectra were obtained as CDCl₃ solutions (reported in ppm), using chloroform as the reference standard (7.27 ppm for the proton and 77.00 ppm for carbon), CD₃OD (3.4 and 4.8 ppm for the protons and 49.3 ppm for carbon), DMSO-d₆ (2.49 ppm for proton), or internally tetramethylsilane (0.00 ppm) when appropriate. Other NMR solvents were used as needed. When peak multiplicities are reported, the following abbreviations are used: s (singlet), d (doublet), t (triplet), q (quartet), m (multiplet), br (broadened), bs (broad singlet), dd (doublet of doublets), dt (doublet of triplets). Coupling constants, when given, are reported in Hertz (Hz).

Infrared (IR) spectra were recorded on an ATR FT-IR Spectrometer as neat oils or solids, and when given are reported in wavenumbers (cm⁻¹). Mass spectra reported are (+)-ES or APCI (+) LC/MS conducted by the Analytical Chemistry Department of Anadys Pharmaceuticals, Inc. Elemental analyses were conducted by the Atlantic Microlab, Inc. in Norcross, Ga. Melting points (mp) were determined on an open capillary apparatus, and are uncorrected.

The described synthetic pathways and experimental procedures utilize many common chemical abbreviations, 2,2-DMP (2,2-dimethoxypropane), Ac (acetyl), ACN (acetonitrile), Bn (benzyl), BnOH (benzyl alcohol), Boc (tert-butoxycarbonyl), Boc₂O (di-tert-butyl dicarbonate), Bz (benzoyl), CSA (camphorsulfonic acid), CSI (chlorosulfonyl isocyanate), DBU (1,8-diazabicyclo[5,4,0]undec-7-ene), DCC(N,N′-dicyclohexylcarbodiimide), DCE (1,2-dichloroethane), DCM (dichloromethane), DEAD (diethylazodicarboxylate), DIBAL (diisobutylaluminum hydride), DIEA (diisopropylethylamine), DMA (N,N-dimethylacetamide), DMAP (4-(N,N-dimethylamino)pyridine), DMF (N,N-dimethylformamide), DMSO (dimethyl sulfoxide), EDC (1-(3-dimethylaminopropyl)-3-ethylcarbodiimide hydrochloride), Et (ethyl), EtOAc (ethyl acetate), EtOH (ethanol), HATU (O-(7-azabenzotriazol-1-yl)-1,1,3,3-tetramethyluronium hexafluorophosphate), HBTU (O-benzotriazol-1-yl-N,N,N′,N′-tetramethyluronium hexafluorophosphate), HF (hydrogen fluoride), HOAc (acetic acid), HOBT (1-hydroxybenzotriazole hydrate), HPLC (high pressure liquid chromatography), IPA (isopropyl alcohol), KHMDS (potassium bis(trimethylsilyl)amide), KN(TMS)₂ (potassium bis(trimethylsilyl)amide), KO^{t}Bu (potassium tert-butoxide), LDA (lithium diisopropylamine), MCPBA (3-chloroperbenzoic acid), Me (methyl), MeCN (acetonitrile), MeOH (methanol), NaCNBH₃ (sodium cyanoborohydride), NaH (sodium hydride), NaN(TMS)₂ (sodium bis(trimethylsilyl)amide), NaOAc (sodium acetate), NaOEt (sodium ethoxide), NEt₃ (triethylamine), NMM (N-methylmorpholine), Phe (phenylalanine), PPTS (pyridinium p-toluenesulfonate), PS (polymer supported), Py (pyridine), pyBOP (benzotriazol-1-yloxy)tripyrrolidinophosphonium hexafluorophosphate), TEA (triethylamine), TFA (trifluoroacetic acid), TFAA (trifluoroacetic anhydride), THF (tetrahydrofuran), TLC (thin layer chromatography), Tol (toluoyl), Val (valine), and the like.

Scheme 1 provides a general procedure that can be used to prepare saturated compounds of Formula I.

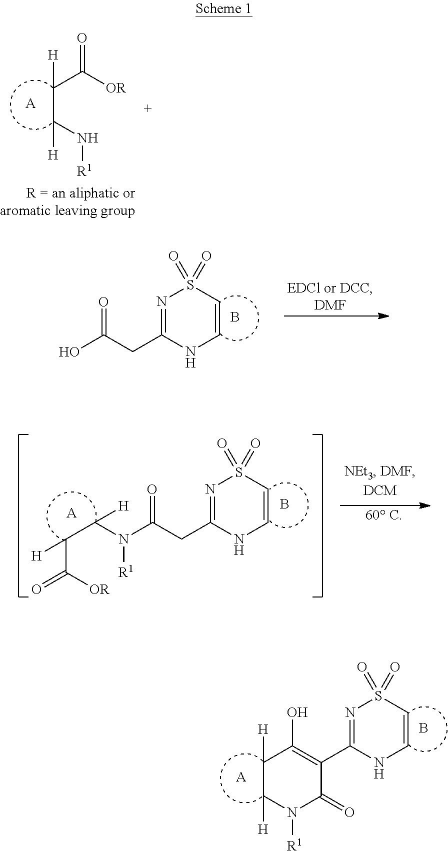

The saturated bridged polycyclic N-substituted-β-amino acid ester intermediates, which can be obtained as described by one of the methods in Schemes 2-4 can be condensed with the appropriate carboxylic acid intermediate (or a salt thereof, e.g., sodium salt) (see US 2010/0034773A1 and US 2009/0306057A1) using standard peptide coupling conditions used for the formation of amide bonds, such as EDCI or DCC, to yield the shown amide. This intermediate can be cyclized with or without isolation in the presence of a base (e.g., triethylamine) to give the desired saturated [1,2,4]thiadiazine 1,1-dioxide compounds.

Scheme 2 provides a general procedure that can be used to prepare bridged polycyclic N-substituted-β-amino acid ester intermediates from anhydrides.

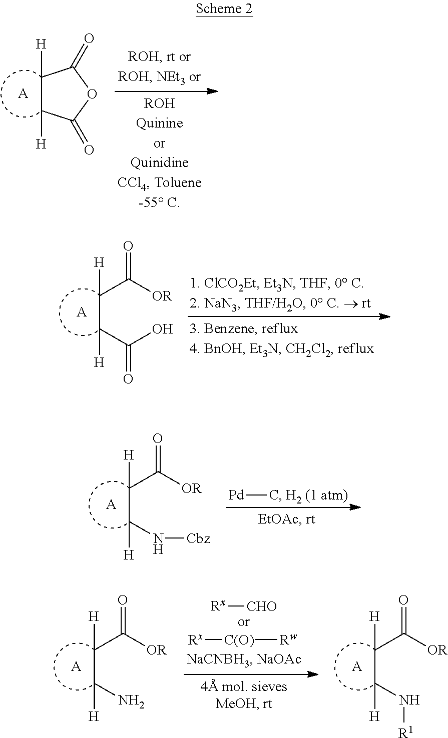

Saturated bridged polycyclic meso-anhydrides can be desymmetrized with alcohols to form the corresponding achiral α,β-cis dicarboxylic monoester intermediates or the chiral α,β-cis dicarboxylic monoester employing enzymes or chiral reagents, such as cinchona alkaloids (e.g., quinine or quinidine) as described in the literature to provide optically active saturated cyclic dicarboxylic acid monoesters (with R as defined in Scheme 1). See J. Org. Chem., 65, 6984-6991 (2000); Synthesis, 11, 1719-1730 (2001), and references cited therein.

These intermediates can be further elaborated into protected saturated bridged polycyclic β-amino acid esters (e.g., Cbz-protected) via a rearrangement reaction, such as the Curtius rearrangement (shown) or a Hofmann degradation. Hydrogenation of the protected saturated bridged polycyclic β-amino acid esters under standard conditions can be used to remove the protecting group and furnish the optically active saturated bridged polycyclic β-amino acid esters, which can be isolated (and used) as either the free bases or their corresponding salts. The optically active saturated bridged polycyclic β-amino acid esters (or their salts) can then be treated with aldehydes or ketones, where R^{x} and R^{w} are independently C₁-C₅ alkyl, C₃-C₈ cycloalkyl, —C₁-C₅ alkylene(C₃-C₈ cycloalkyl), —C₁-C₅ alkylene(aryl), —C₁-C₅ alkylene(heterocyclyl), aryl, or heterocyclyl, or R^{w} can combine with R^{x} to form a 3- to 8-membered ring, in the presence of a reducing agent (such as sodium cyanoborohydride) to afford the desired optically active saturated cyclic N-substituted-β-amino acid ester intermediates. Alternatively, the reaction sequence described above can be performed without enzymes or chiral reagents leading to the corresponding achiral intermediates and products.

In certain cases, unprotected bridged polycyclic β-amino acid esters can also be prepared directly from the α,β-cis dicarboxylic monoester, which can further be elaborated to the bridged polycyclic N-substituted-β-amino acid ester. Scheme 3 provides a general procedure that can be used to prepare bridged polycyclic N-substituted-β-amino acid ester intermediates from anhydrides.

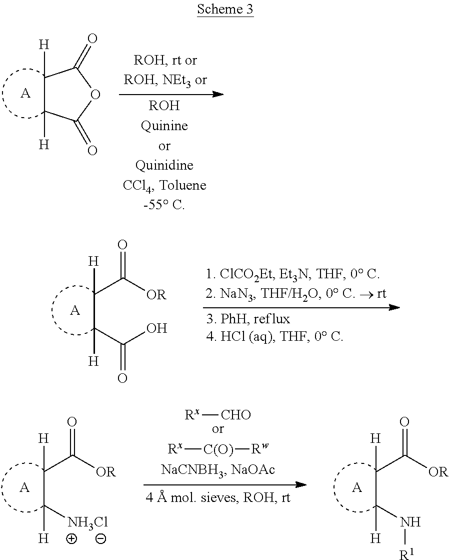

Scheme 4 provides an alternate general procedure that can be used to prepare saturated bridged polycyclic N-substituted-β-amino acid ester intermediates.

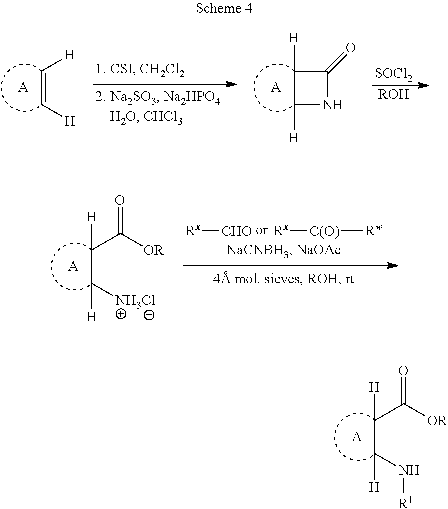

Bridged polycyclic olefins can be reacted with chlorosulfonyl isocyanate to yield β-lactams. These intermediates can be hydrolyzed directly to corresponding β-amino acids ester hydrochloric acid salts using thionyl chloride and the appropriate alcohol. The bridged polycyclic β-amino acid esters can then be treated with aldehydes or ketones (with R^{x} and R^{w} as defined in Scheme 2) in the presence of a reducing agent, such as sodium cyanoborohydride, to afford the desired bridged polycyclic N-substituted-β-amino acid ester intermediates.

Optically active saturated bridged polycyclic β-amino acid esters can also be generated by resolving the diastereomeric salts formed from optically pure acids such as (1S)-(+)- or (1R)-(−)-10-camphorsulfonic acid, (R)-(−)- or (S)-(+)-mandelic acid, (2R,3R)-(+)- or (2S,3S)-(−)-tartaric acid, and (S)-(−)- or (R)-(+)-malic acid.

The optically pure bridged polycyclic β-amino acid esters can subsequently be N-substituted and further transformed to optically active forms of compounds disclosed in Formula I as described in the Schemes above.

Scheme 5 provides a specific procedure that was used to prepare rac-N-{3-[(4S,9R)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide or rac-N-({3-[(4S,9R)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide from cyclic anhydride 6-oxahexacyclo[7.5.0.0^{2,14}.0^{3,11}.0^{4,8}.0^{10,12}]tetradecane-5,7-dione.

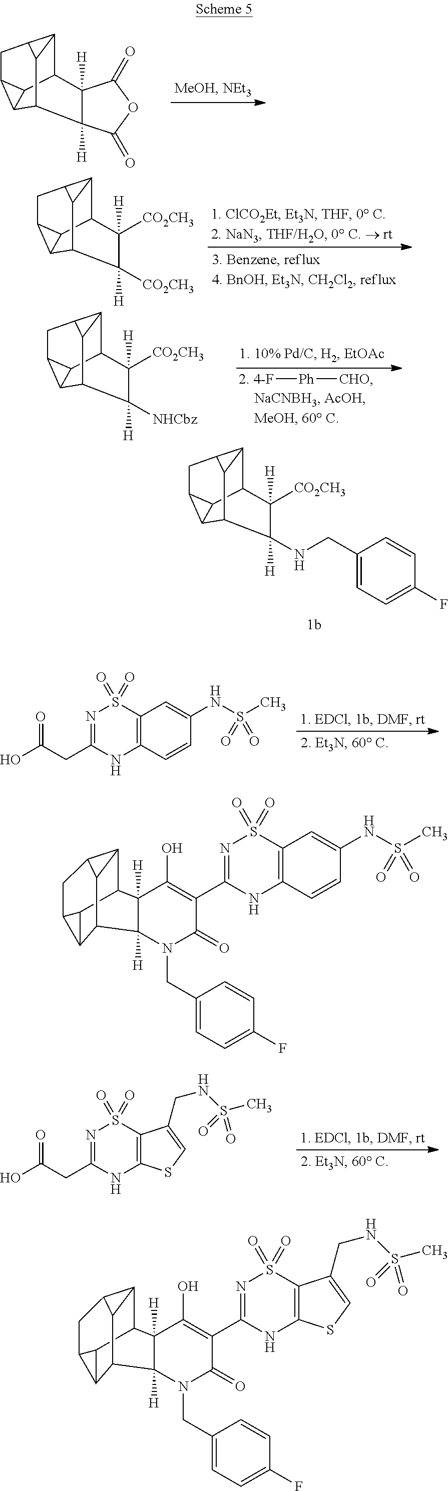

The symmetrical anhydride 6-oxahexacyclo[7.5.0.0^{2,14}.0^{3,11}.0^{4,8}.0^{10,12}]tetradecane-5,7-dione (prepared as described in Chem. Ber. 1983, 116, p. 587-609) was opened to the racemic cis α,β-dicarboxylic acid mono-methyl ester with methanol under basic conditions. The carboxylic acid was converted sequentially to the mixed anhydride with ethyl chloroformate and the corresponding acyl azide with sodium azide. Without purification, a benzene solution of this acyl azide was carefully subjected to heat which induces the acyl azide to rearrange to the isocynate, and the Cbz group was formed with benzyl alcohol. The Cbz group was subjected to catalytic hydrogenation conditions to produce the racemic cis-β-amino acid methyl ester. Using reductive alkylation conditions with sodium cyanoborohydride and 4-fluoro-benzaldehyde, the free amine was alkylated to give intermediate 1b. Intermediate 1b was then separately converted to rac-N-{3-[(4S,9R)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide with 7-methanesulfonylamino-1,1-dioxo-1,4-dihydro-1λ⁶-benzo[1,2,4]thiadiazin-3-yl)-acetic acid (prepared as described in (US 2010/0034773A1) and rac-N-({3-[(4S,9R)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide with [7-(methanesulfonylamino-methyl)-1,1-dioxo-1,4-dihydro-1λ⁶-thieno[2,3-e][1,2,4]thiadiazin-3-yl]-acetic acid (prepared as described in US 2009/0306057A1) via amide bond formation and cyclization.

Purification of rac-N-{3-[(4S,9R)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide by chiral HPLC was used to separate N-{3-[(4S,9R)-5-[(4-fluorophenyl)methyl]-8-hydroxy-6-oxo-5-azahexacyclo[8.5.0.0^{2,15}.0^{3,12}.0^{11,13}]pentadec-7-en-7-yl]-1,1-dioxo-4H-1λ⁶,2,4-benzothiadiazin-7-yl}methanesulfonamide from N-{3-[(4R,9S)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide.

Scheme 6 provides a specific procedure that was used to prepare rac-N-{3-[(2S,7R)-3-[(4-fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,2,4-benzothiadiazin-7-yl}methanesulfonamide and rac-N-({3-[(2S,7R)-3-[(4-fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,5,2,4-thieno[2,3-e][1λ⁶,2,4]thiadiazin-7-yl}methyl)methanesulfonamide from rac-(2S,5R)-3-azatetracyclo[6.3.1.1^{6,10}.0^{2,5}]tridecan-4-one.

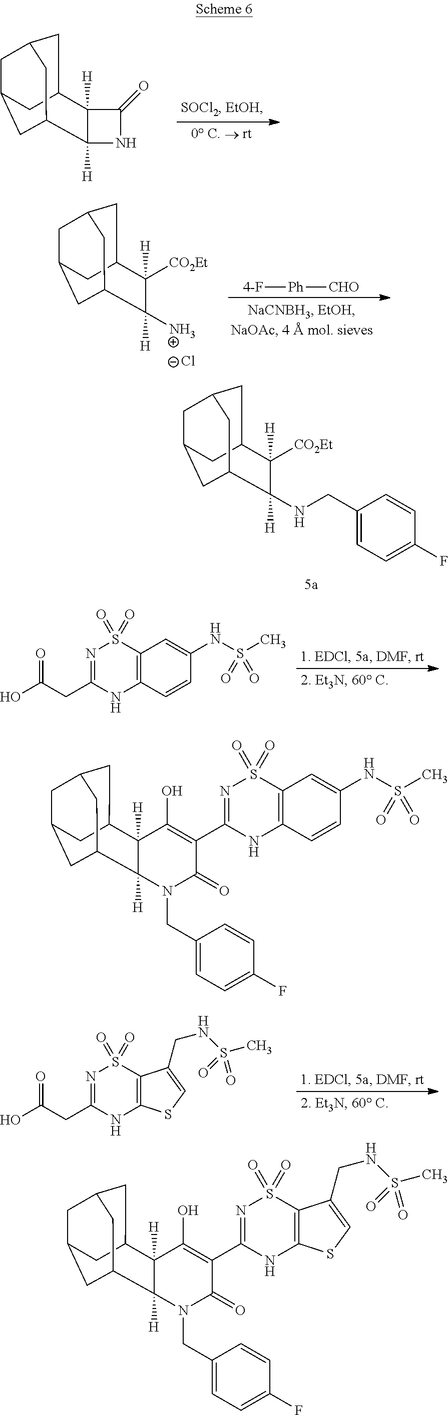

An anhydrous solution of hydrochloric acid in ethanol was carefully prepared from thionyl chloride and then added to rac-(2S,5R)-3-azatetracyclo[6.3.1.1^{6,10}.0^{2,5}]tridecan-4-one (prepared as described in Adv. Synth. Catal. 2004, 346, p. 566-572) to cleave the β-lactam and form the HCl salt of the cis racemic ethyl ester of (4R,5S)-5-amino-tricyclo[4.3.1.1^{3,8}]undecane-4-carboxylic acid. This material was directly subjected to reductive alkylation conditions with 4-fluorobenzaldehyde and sodium cyanoborohydride to give intermediate 5a.

Scheme 7 provides a specific procedure that was used to prepare rac-N-{3-[(1R,9S)-10-[(4-fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide. Intermediate 5a was then separately converted to rac-N-{3-[(2S,7R)-3-[(4-fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,2,4-benzothiadiazin-7-yl}methanesulfonamide with 7-methanesulfonylamino-1,1-dioxo-1,4-dihydro-1λ⁶-benzo[1,2,4]thiadiazin-3-yl)-acetic acid (prepared as described in US 2010/0034773A1) and rac-N-({3-[(2S,7R)-3-[(4-fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,5,2,4-thieno[2,3-e][1λ⁶,2,4]thiadiazin-7-yl}methyl)methanesulfonamide with [7-(methanesulfonylamino-methyl)-1,1-dioxo-1,4-dihydro-1λ⁶-thieno[2,3-e][1,2,4]thiadiazin-3-yl]-acetic acid (prepared as described in US20090306057A1) via amide bond formation and cyclization.

Scheme 7 provides a specific procedure that was used to prepare rac-N-{3-[(1R,9S)-10-[(4-fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide.

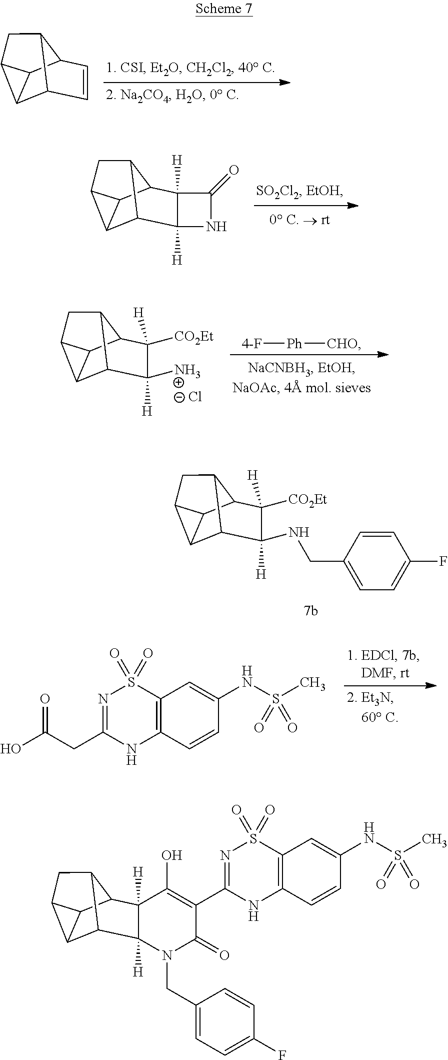

Chlorosulfonyl isocyanate was used to convert the alkene bond of tetracyclo[4.3.0.0^{2,4}.0^{3,7}]non-8-ene (prepared as described in J. Am. Chem. Soc. 1995, 117, p. 10276-10291) to the β-lactam of rac-(3S,6R)-4-azapentacyclo[5.4.0.0^{2,9}. 0^{3,6}.0^{8,10}]undecan-5-one. An anhydrous solution of hydrochloric acid in ethanol, prepared from thionyl chloride was then used to open this β-lactam to afford the HCl salt of the cis racemic ethyl ester of (8R,9S)-9-amino-tetracyclo[4.3.0.0^{2,4}.0^{3,7}]nonane-8-carboxylic acid. This compound was directly exposed to reductive alkylation conditions with 4-fluorobenzaldehyde and sodium cyanoborohydride to produce intermediate 7b. Amino ester 7b was combined with 7-methanesulfonylamino-1,1-dioxo-1,4-dihydro-1λ⁶-benzo[1,2,4]thiadiazin-3-yl)-acetic acid (prepared as described in US 2010/0034773A1) under amide forming conditions with EDCI followed by ring formation with triethylamine and mild heating to give the final product, rac-N-{3-[(1R,9S)-10-[(4-fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide.

Scheme 8 illustrates a specific procedure to synthesize rac-N-{3-[(1R,9S)-10-[(4-fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide and rac-N-({3-[(1R,9S)-10-[(4-fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,5,2,4-thieno[2,3-e][1λ⁶,2,4]thiadiazin-7-yl}methyl)methanesulfonamide.

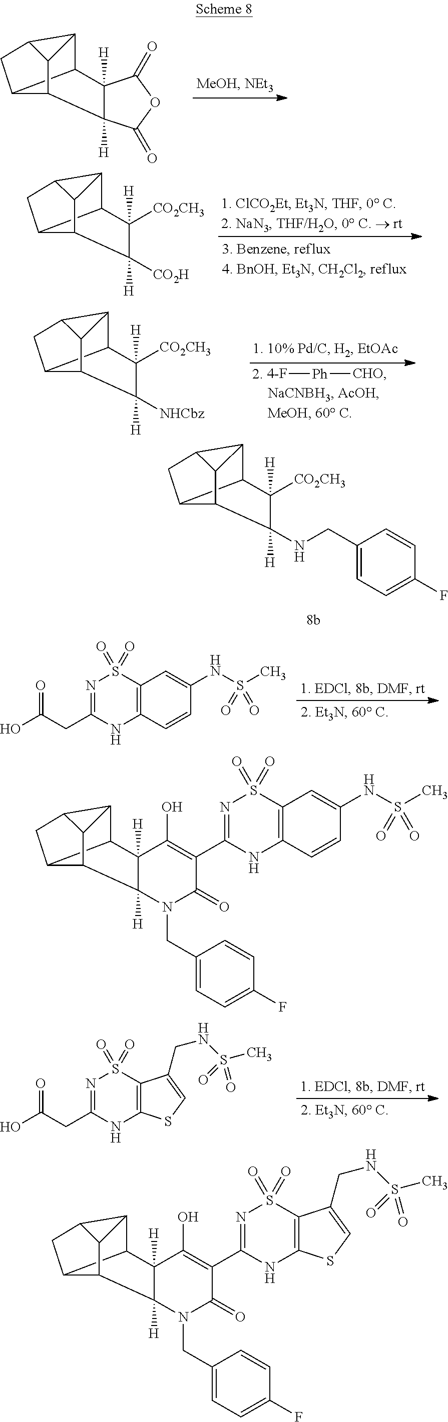

Cyclic anhydride 5-oxapentacyclo[6.4.0.0^{2,10}.0^{3,7}.0^{9,11}]dodecane-4,6-dione (prepared as described in J. Chem. Soc. 1964, p. 5416-5421) was opened to the corresponding racemic cis α,β-dicarboxylic acid mono-methyl ester when dissolved in anhydrous methanol and triethylamine. The carboxylic acid function was converted sequentially to the mixed anhydride with ethyl chloroformate and the corresponding acyl azide with sodium azide. Without purification, a benzene solution of this acyl azide was carefully subjected to heat which induces the acyl azide to rearrange to the isocynate, and the Cbz group was formed by addition of benzyl alcohol. The Cbz group was subjected to catalytic hydrogenation conditions to produce the racemic cis-β-amino acid methyl ester. Using reductive alkylation conditions with sodium cyanoborohydride and 4-fluoro-benzaldehyde, the free amine was alkylated to give intermediate 8b. Intermediate 8b was then separately converted to synthesize rac-N-{3-[(1R,9S)-10-[(4-fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide and rac-N-({3-[(1R,9S)-10-[(4-fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,5,2,4-thieno[2,3-e][1λ⁶,2,4]thiadiazin-7-yl}methyl)methanesulfonamide with 7-methanesulfonylamino-1,1-dioxo-1,4-dihydro-1λ⁶-benzo[1,2,4]thiadiazin-3-yl)-acetic acid (prepared as described in US 2010/0034773A1) and rac-N-({3-[(4S,9R)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide with [7-(methanesulfonylamino-methyl)-1,1-dioxo-1,4-dihydro-1λ⁶-thieno[2,3-e][1,2,4]thiadiazin-3-yl]-acetic acid (prepared as described in US 2009/0306057A1) via amide bond formation and cyclization.

Scheme 9 illustrates a specific procedure to synthesize rac-N-{3-[(1R,10S)-11-[(4-fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide and rac-N-({3-[(1R,10S)-11-[(4-fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,5,2,4-thieno[2,3-e][1λ⁶,2,4]thiadiazin-7-yl}methyl)methanesulfonamide.

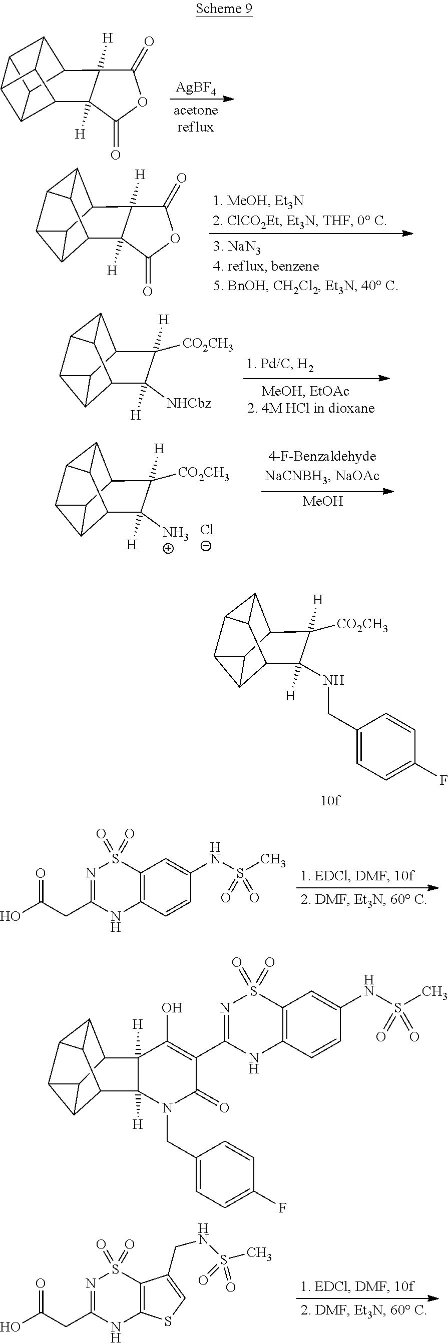

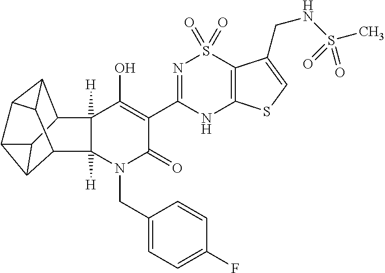

The symmetrical anhydride 6-oxahexacyclo[7.4.0.0^{2,12}.0^{3,11}.0^{4,8}.0^{10,13}]tridecane-5,7-dione (prepared as described in J. Am. Chem. Soc. 1991, 113, p. 7882-7886 and J. Am. Chem. Soc. 1971, 93, p. 2459-2463) was subjected to cationic rearrangement conditions with silver tetrafluoroborate to give the symmetrical cyclic anhydride 6-oxahexacyclo[7.4.0.0^{2,13}.0^{3,11}.0^{4,8}.0^{10,12}]tridecane-5,7-dione. The anhydride was opened to the racemic cis α,β-dicarboxylic acid mono-methyl ester with methanol under basic conditions. The carboxylic acid was further converted to the mixed anhydride with ethyl chloroformate and then to the corresponding acyl azide with sodium azide. Without purification, a benzene solution of this acyl azide was carefully subjected to heat which induces the acyl azide to rearrange to the isocynate, and the Cbz group of rac-methyl (9R,10S)-10-{[(benzyloxy)carbonyl]amino)pentacyclo[4.4.0.0^{2,4}.0^{3,8}.0^{5,7}]decane-9-carboxylate was formed with benzyl alcohol. Under catalytic hydrogenation conditions the Cbz group was removed and the HCl salt of this racemic cis-β-amino acid methyl ester was formed using anhydrous hydrogen chloride in 1,4-dioxane. Utilizing reductive alkylation conditions with sodium cyanoborohydride and 4-fluoro-benzaldehyde, the primary amine was alkylated to give intermediate 10f. Intermediate 10f was then separately converted to rac-N-{3-[(1R,10S)-11-[(4-fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl)methanesulfonamide with 7-methanesulfonylamino-1,1-dioxo-1,4-dihydro-1λ⁶-benzo[1,2,4]thiadiazin-3-yl)-acetic acid (prepared as described in US 2010/0034773A1) and rac-N-({3-[(1R,10S)-11-[(4-fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,5,2,4-thieno[2,3-e][1λ⁶,2,4]thiadiazin-7-yl}methyl)methanesulfonamide with [7-(methanesulfonylamino-methyl)-1,1-dioxo-1,4-dihydro-1λ⁶-thieno[2,3-e][1,2,4]thiadiazin-3-yl]-acetic acid (prepared as described in US 2009/0306057A1) via amide bond formation and cyclization.

Scheme 10 outlines specific synthetic procedure to prepare rac-N-{3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,2,4-benzothiadiazin-7-yl}methanesulfonamide and rac-N-({3-[(1R,10S)-11-[(4-fluorophenyl)methyl]-14-hydroxy-12-oxo-11-azahexacyclo[8.4.0.0^{2,5}. 0^{3,8}.0^{4,7}.0^{6,9}]tetradec-13-en-13-yl]-1,1-dioxo-4H-1λ⁶,5,2,4-thieno[2,3-e][1λ⁶,2,4]thiadiazin-7-yl}methyl)methanesulfonamide.

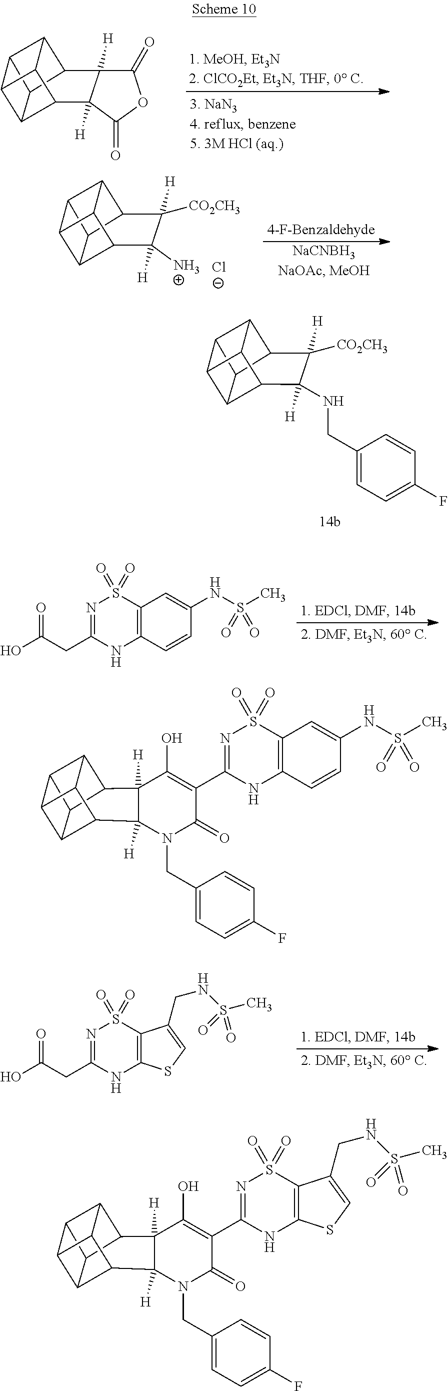

The symmetrical anhydride 6-oxahexacyclo[7.4.0.0^{2,12}.0^{3,11}.0^{4,8}.0^{10,13}]tridecane-5,7-dione was subjected to methanol under basic conditions to provide the racemic cis α,β-dicarboxylic acid mono-methyl ester. The carboxylic acid was further converted to the mixed anhydride with ethyl chloroformate and then to the corresponding acyl azide with an aqueous solution of sodium azide. Without purification, a benzene solution of this acyl azide was carefully refluxed causing the acyl azide to rearrange to the isocynate, which was subjected to aqueous hydrochloric acid to form the HCl salt of rac-methyl (9R,10S)-10-aminopentacyclo[4.4.0.0^{2,5}.0^{3,8}.0^{4,7}]decane-9-carboxylate. Using reductive alkylation conditions with sodium cyanoborohydride and 4-fluoro-benzaldehyde, the primary amine was alkylated to give intermediate 14b. Intermediate 14b was then separately converted to final products rac-N-{3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,2,4-benzothiadiazin-7-yl}methanesulfonamide with 7-methanesulfonylamino-1,1-dioxo-1,4-dihydro-1λ⁶-benzo[1,2,4]thiadiazin-3-yl)-acetic acid and rac-N-({3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,5,2,4-thieno[2,3-e][1λ⁶,2,4]thiadiazin-7-yl}methyl)methanesulfonamide with [7-(methanesulfonylamino-methyl)-1,1-dioxo-1,4-dihydro-1λ⁶-thieno[2,3-e][1,2,4]thiadiazin-3-yl]-acetic acid via amide bond formation and ring formation.

Scheme 11 provides procedures that can used to prepare rac-N-{3-[(4S,9R)-14,14-difluoro-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide or rac-N-({3-[(4S,9R)-14,14-difluoro-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide.

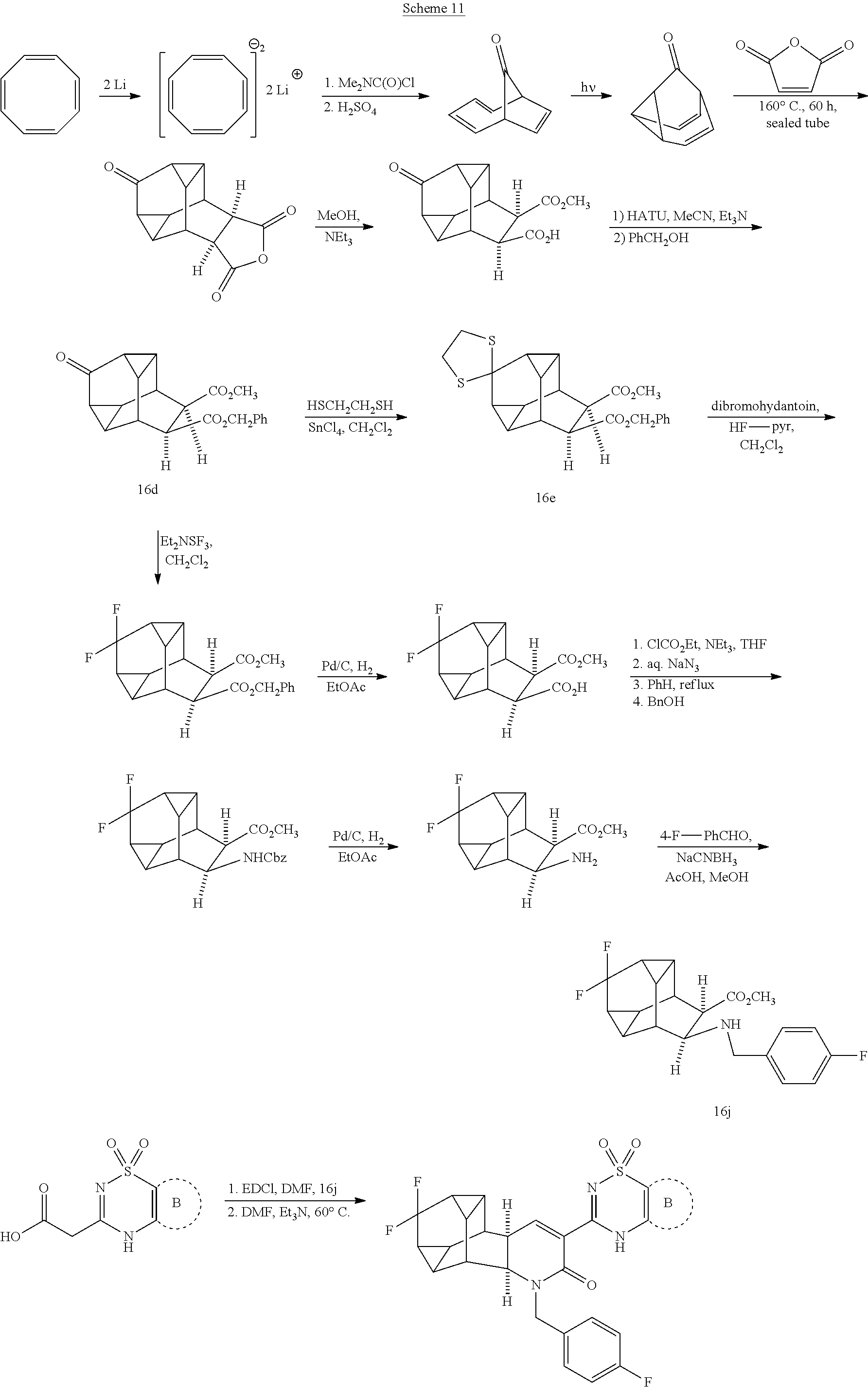

Following procedures described in J. Org. Chem. Soc. 1972, 37, p. 2517-2519 and J. Am. Chem. Soc. 1972, 94, p. 5366-5373, bicyclo[4.2.1]nona-2,4,7-trien-9-one was prepared from the lithium dianion of 1,3,5,7-cyclooctatetrene and dimethyl carbamoyl chloride. Photolysis of this keto-triene induced a sigmatropic rearrangement to give tricyclo[3.3.1.0^{2,8}]nona-3,6-dien-9-one. Following the procedure in Helvetica Chemica Acta, 1990, 73, p. 1182-1196, this dien-one was converted to 6-oxahexacyclo[7.5.0.0^{2,14}.0^{3,11}.0^{4,8}.0^{10,12}]tetradecane-5,7,13-trione with maleic anhydride. This cyclic anhydride was then be exposed to anhydrous methanol and triethylamine to give the corresponding racemic cis α,β-dicarboxylic acid mono-methyl ester. The carboxylic acid was esterified with benzyl alcohol using 2-(7-Aza-1H-benzotriazole-1-yl)-1,1,3,3-tetramethyluronium hexafluorophosphate to yield afforded mixed diester 16d of the racemic-cis α,β-dicarboxylic acid. The ketone 16d was directly converted under anhydrous conditions with ethane-1,2-dithiol and a Lewis acid to dithiolane 16e. The ketone function of polycyclic diester 16d can be transformed to a geminal difluoride group with diethylaminosulfur trifluoride (DAST) or other appropriate fluorinating reagents.

Alternately the dithiolane group of polycyclic diester 16e can also be converted to a geminal difluoride group under reported conditions using hydrofluoric acid (J. Org. Chem. 1986, Vol. 51, No. 18, p. 3508-3513). Catalytic hydrogenation can be used to remove the benzyl ester of rac-10-benzyl-11-methyl (10S,11R)-5-oxopentacyclo[5.4.0.0^{2,4}.0^{3,9}.0^{6,8}]undecane-10,11-dicarboxylate and the free acid can then be converted to the corresponding racemic cis-β-amino acid methyl ester through a series of standard synthetic transformations commonly used in other examples described above. Utilizing reductive alkylation conditions with sodium cyanoborohydride and 4-fluoro-benzaldehyde, the free amine can be alkylated to give intermediate 16j. Intermediate 16j can then be separately converted to rac-N-{3-[(4S,9R)-14,14-difluoro-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide with 7-methanesulfonylamino-1,1-dioxo-1,4-dihydro-1λ⁶-benzo[1,2,4]thiadiazin-3-yl)-acetic acid and rac-N-({3-[(4S,9R)-14,14-difluoro-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide with [7-(methanesulfonylamino-methyl)-1,1-dioxo-1,4-dihydro-1λ⁶-thieno[2,3-e][1,2,4]thiadiazin-3-yl]-acetic acid via amide bond formation and ring closure.

Scheme 12 provides procedures that can used to prepare rac-N-{3-[(4S,9R)-5-[(4-fluorophenyl)methyl]-8-hydroxy-6,14-dioxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide or rac-N-({3-[(4S,9R)-5-[(4-fluorophenyl)methyl]-8-hydroxy-6,14-dioxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide.

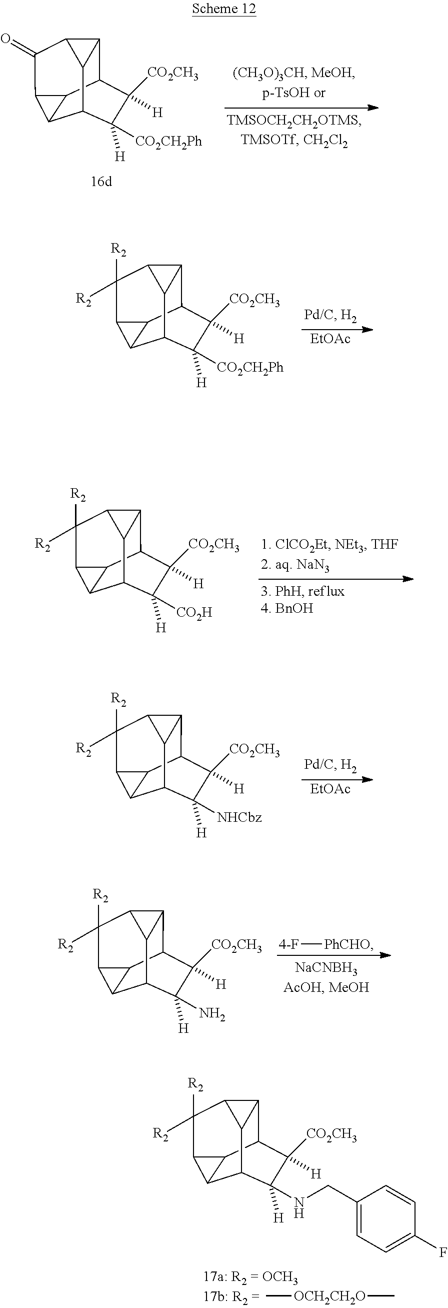

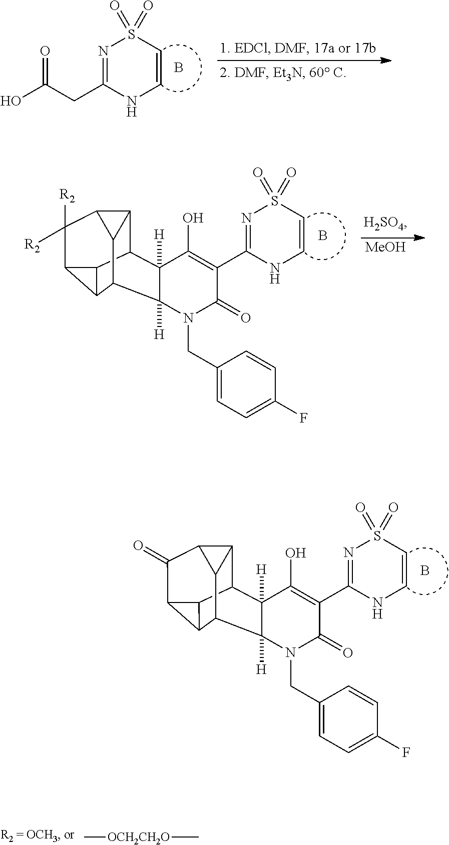

Beginning with mixed diester 16d, described in Scheme 11, the ketone can be protected as the dimethyl ketal or the spiro-dioxalane ketal using common procedures. The benzyl ester can be selectively removed under catalytic hydrogenation conditions to form the racemic cis α,β-dicarboxylic acid mono-methyl ester. The free acid can then be converted to the corresponding racemic cis-β-amino acid methyl ester through a series of standard synthetic transformations commonly used in other examples described above. Utilizing reductive alkylation conditions with sodium cyanoborohydride and 4-fluoro-benzaldehyde, the free amine can be alkylated to give intermediate 17a or 17b. Intermediate 17a or 17b can then be separately converted to rac-N-{3-[(4S,9R)-14,14-dimethoxy-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide or rac-N-{3-[(4S,9R)-5′-[(4-fluorophenyl)methyl]-8′-hydroxy-6′-oxo-5′-azaspiro[1,3-dioxolane-2,14′-hexacyclo[8.5.0.0^{2,15}.0^{3,12}.0^{4,9}.0^{11,13}]pentadec-7′-en-7′-yl]-1,1-dioxo-4H-1λ⁶,2,4-benzothiadiazin-7-yl}methanesulfonamide product with 7-methanesulfonylamino-1,1-dioxo-1,4-dihydro-1λ⁶-benzo[1,2,4]thiadiazin-3-yl)-acetic acid. Intermediate 17a or 17b can also be separately converted and rac-N-({3-[(4S,9R)-14,14-dimethoxy-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide or rac-N-{3-[(4S,9R)-5′-[(4-fluorophenyl)methyl]-8′-hydroxy-6′-oxo-5′-azaspiro[1,3-dioxolane-2,14′-hexacyclo[8.5.0.0^{2,15}.0^{3,12}.0^{4,9}.0^{11,13}]pentadec-7′-en-7′-yl]-1,1-dioxo-4H-1λ⁶,5,2,4-thieno[2,3-e][1λ⁶,2,4]thiadiazin-7-yl}methyl)methanesulfonamide with [7-(methanesulfonylamino-methyl)-1,1-dioxo-1,4-dihydro-1λ⁶-thieno[2,3-e][1,2,4]thiadiazin-3-yl]-acetic acid. The ketal products can then separately converted to rac-N-{3-[(4S,9R)-5-[(4-fluorophenyl)methyl]-8-hydroxy-6,14-dioxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide and rac-N-({3-[(4S,9R)-5-[(4-fluorophenyl)methyl]-8-hydroxy-6,14-dioxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide using aqueous sulfuric acid and an appropriate solvent.

### Example 1

### rac-N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide

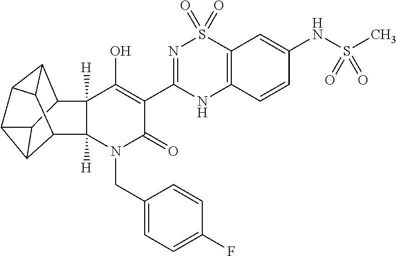

### (a) rac-Methyl (10R,11S)-11-{[(benzyloxy)carbonyl]amino}pentacyclo[5.4.0.0^{2,4}.0^{3,9}.0^{6,8}]undecane-10-carboxylate

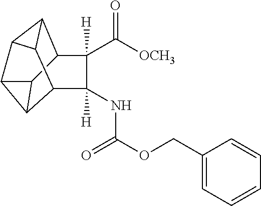

6-Oxahexacyclo[7.5.0.0^{2,14}.0^{3,11}.0^{4,8}.0^{10,12}]tetradecane-5,7-dione (prepared as described in Chem. Ber. 1983, 116, 587-609, 60 mg, 0.28 mmol) was dissolved in triethylamine (1 mL) and methanol (10 mL). The reaction was stirred at room temperature overnight. The mixture was concentrated and used directly for next step. The crude was dissolved in tetrahydrofuran (2 mL) and cooled to 0° C. Triethylamine (0.11 mL, 0.78 mmol) was added followed by the dropwise addition of ethyl chloroformate (0.05 mL, 0.52 mmol). The reaction was stirred at 0° C. for 1 h. A solution of sodium azide (68 mg, 1.04 mmol) in water (0.4 mL) was added to the reaction at 0° C. The reaction was stirred at 0° C. for 5 min, then at room temperature for 2 h. The mixture was diluted with water (5 mL) and half saturated sodium bicarbonate solution (5 mL). The mixture was extracted with ethyl acetate (10 mL×3). The organics were combined, dried over sodium sulfate, filtered, and concentrated to afford a clear oil. The oil was dissolved in benzene (2 mL) and refluxed for 2 h. Upon cooling to room temperature the solution was concentrated and dissolved in dichloromethane (2 mL). Benzyl alcohol (0.03 mL, 0.29 mmol) was added followed by triethylamine (0.07 mL, 0.52 mmol). The reaction was refluxed overnight. Upon cooling to room temperature the mixture was concentrated to afford a golden oil. Purification by flash column chromatography (Merck silica gel 60, 40-63 μm, 0-15% ethyl acetate in hexanes) afforded the desired product, rac-methyl (10R,11S)-11-{[(benzyloxy)carbonyl]amino}pentacyclo[5.4.0.0^{2,40}.0^{3,9}.0^{6,8}]undecane-10-carboxylate (42 mg, 0.12 mmol, 57%), as a clear oil. ¹H NMR (400 MHz, CDCl₃) δ: 0.68-2.99 (11H, m), 3.58 (3H, s), 4.18 (1H, dt, J₁=8.0 Hz, J₂=2.2 Hz), 5.10 (2H, dd, J=18.1 Hz, J₂=9.9 Hz), 6.00-6.02 (1H, m), 7.30-7.38 (5H, m). LC-MS (ESI) calcd for C₂₁H₂₃NO₄ 353.41, found 354.4 [M+H⁺].

### (b) rac-Methyl (10R,11S)-11-{[(4-fluorophenyl)methyl]amino}pentacyclo[5.4.0.0^{2,40}.0^{3,9}.0^{6,8}]undecane-10-carboxylate

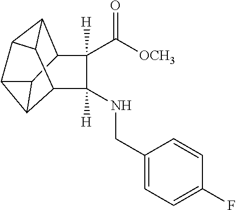

rac-Methyl (10R,11S)-11-{[(benzyloxy)carbonyl]amino}pentacyclo[5.4.0.0^{2,4}.0^{3,9}.0^{6,8}]undecane-10-carboxylate (42 mg, 0.12 mmol) was dissolved in ethyl acetate (5 mL), followed by the addition of 10% palladium on carbon (8 mg). The reaction was purged with hydrogen and was stirred under hydrogen atmosphere overnight. The mixture was filtered through a pad of celite and concentrated. The crude amine was used directly for the next step.

The crude amine was dissolved in methanol (1 mL), followed by the sequential addition of 4-fluoro-benzaldehyde (0.01 mL, 0.09 mmol), acetic acid (0.01 mL, 0.18 mmol) and sodium cyanoborohydride (12 mg, 0.18 mmol). The reaction was stirred at 60° C. overnight. The reaction was cooled to room temperature, diluted with saturated aqueous sodium bicarbonate solution (4 mL) and stirred for additional 10 min. The mixture was extracted with ethyl acetate (4 mL×3). The organics were combined, dried over sodium sulfate, filtered and concentrated. Purification by flash column chromatography (Merck silica gel 60, 40-63 μm, 0-20% ethyl acetate in hexanes) afforded the desired product, rac-methyl (10R,11S)-11-{[(4-fluorophenyl)methyl]amino}pentacyclo[5.4.0.0^{2,40}.0^{3,9}.0^{6,8}]undecane-10-carboxylate (24 mg, 0.07 mmol, 58%), as a clear oil. LC-MS (ESI) calcd for C₂₀H₂₂FNO₂ 327.39, found 328.4 [M+H⁺].

### (c) rac-N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide

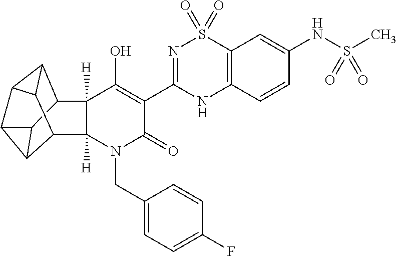

(7-Methanesulfonylamino-1,1-dioxo-1,4-dihydro-1λ⁶-benzo[1,2,4]thiadiazin-3-yl)-acetic acid (prepared as described in US 2010/0034773A1) (23 mg, 0.07 mmol) was dissolved in N,N-dimethylformamide (1 mL). rac-Methyl (10R,11S)-11-{[(4-fluorophenyl)methyl]amino}pentacyclo[5.4.0.0^{2,4}.0^{3,9}.0^{6,8}]undecane-10-carboxylate (24 mg, 0.07 mmol) was added followed by the addition of N-(3-dimethylaminopropyl)-N′-ethylcarbodiimide hydrochloride (21 mg, 0.11 mmol). The reaction was stirred at room temperature for 1 h. Triethylamine (0.06 mL, 0.42 mmol) was added and the mixture was stirred at 60° C. overnight. Upon cooling, the mixture was poured into 1.0 M aqueous hydrochloric acid solution (5 mL). The mixture was extracted with ethyl acetate (5 mL×3). The organics were combined, dried over sodium sulfate, filtered and concentrated. Purification by flash column chromatography (Merck silica gel 60, 40-63 μm, 0-80% ethyl acetate in hexanes) afforded rac-N-{3-[(4S,9R)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide. (21 mg, 0.04 mmol, 48%), as a white powder. ¹H NMR (400 MHz, Acetone-d₆) δ: 0.74-3.30 (11H, m), 3.10 (3H, s), 3.84-3.93 (1H, m), 4.57-4.66 (1H, m), 5.15-5.18 (1H, m), 7.10-7.80 (7H, m), 14.62 (1H, s). LC-MS (ESI) calcd for C₂₉H₂₇FN₄O₆S₂ 610.68, found 611.2 [M+H⁺].

### Example 2

### N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide

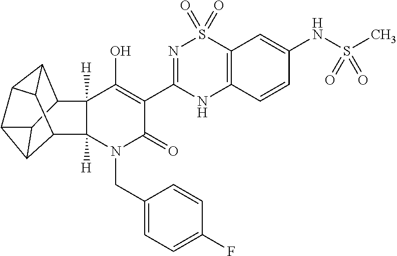

Chiral separation of rac-N-{3-[(4S,9R)-5-[(4-fluorophenyl)methyl]-8-hydroxy-6-oxo-5-azahexacyclo[8.5.0.0^{2,15}.0^{3,12}.0^{11,13}]pentadec-7-en-7-yl]-1,1-dioxo-4H-1λ⁶,2,4-benzothiadiazin-7-yl}methanesulfonamide (18 mg, 0.03 mmol) by HPLC (Chiralpak AS-RH, 4.6×150 mm, 5 micron with mobile phases 18% 0.05% TFA in water and 82% 0.05% TFA in acetonitrile isocratic for 15 min., 0.8 mL/min., 310 nm, Rt=3.3 min) afforded enantiomerically pure N-{3-[(4S,9R)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide (8 mg, 0.01 mmol, 44%) as a white powder. ¹H NMR (400 MHz, Acetone-d₆) δ: 0.74-3.30 (11H, m), 3.10 (3H, s), 3.84-3.93 (1H, m), 4.57-4.66 (1H, m), 5.15-5.18 (1H, m), 7.10-7.80 (7H, m), 14.62 (1H, s). LC-MS (ESI) calcd for C₂₉H₂₇FN₄O₆S₂ 610.68, found 611.2 [M+H⁺].

### Example 3

### N-{3-[(4R,9S)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide

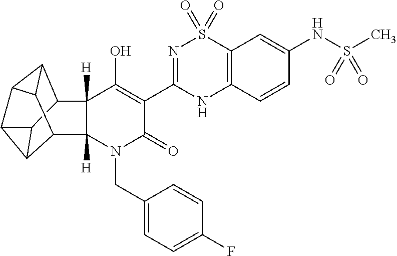

From the same chiral HPLC experiment described in Example 2 above, N-{3-[(4R,9S)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide (t=6.8 min) was isolated as a white powder. (8 mg, 0.01 mmol, 44%) ¹H NMR (400 MHz, Acetone-d₆) δ: 0.74-3.30 (11H, m), 3.10 (3H, s), 3.84-3.93 (1H, m), 4.57-4.66 (1H, m), 5.15-5.18 (1H, m), 7.10-7.80 (7H, m), 14.62 (1H, s). LC-MS (ESI) calcd for C₂₉H₂₇FN₄O₆S₂610.68, found 611.2 [M+H⁺].

### Example 4

### rac-N-({3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide

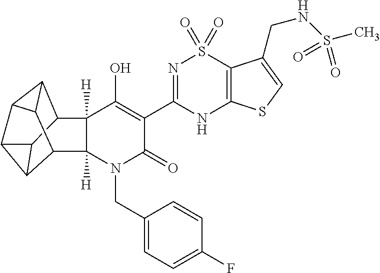

[7-(Methanesulfonylamino-methyl)-1,1-dioxo-1,4-dihydro-1λ⁶-thieno[2,3-e][1,2,4]thiadiazin-3-yl]-acetic acid (prepared as described in US 2009/0306057A1) (32 mg, 0.09 mmol) was dissolved in N,N-dimethylformamide (1 mL). rac-Methyl (10R,11S)-11-{[(4-fluorophenyl)methyl]amino}pentacyclo[5.4.0.0^{2,4}.0^{3,9}.0^{6,8}]undecane-10-carboxylate (prepared as described in Example 1, 29 mg, 0.09 mmol) was added followed by the addition of N-(3-dimethylaminopropyl)-N′-ethylcarbodiimide hydrochloride (27 mg, 0.14 mmol). The reaction was stirred at room temperature for 1 h. Triethylamine (0.08 mL, 0.54 mmol) was added and the mixture was stirred at 60° C. overnight. Upon cooling, the mixture was poured into 1.0 M aqueous hydrochloric acid solution (5 mL). The mixture was extracted with ethyl acetate (5 mL×3). The organics were combined, dried over sodium sulfate, filtered and concentrated. Purification by flash column chromatography (Merck silica gel 60, 40-63 μm, 0-80% ethyl acetate in hexanes) afforded the desired product, rac-N-({3-[(4S,9R)-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide (13 mg, 0.04 mmol, 45%), as a white powder. ¹H NMR (400 MHz, Acetone-d₆) δ: 0.66-3.27 (11H, m), 2.96 (3H, s), 4.42-4.48 (2H, m), 4.56-4.64 (1H, m), 5.01-5.17 (1H, m), 7.09-7.52 (5H, m). LC-MS (ESI) calcd for C₂₈H₂₇FN₄O₆S₃ 630.73, found 631.2 [M+H⁺]

### Example 5

### rac-N-{3-[(2S,7R)-3-[(4-Fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,2,4-benzothiadiazin-7-yl}methanesulfonamide

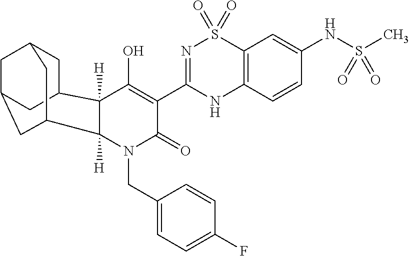

### (a) rac-Ethyl (4R,5S)-5-{[(4-fluorophenyl)methyl]amino}tricyclo[4.3.1.1^{3,8}]undecane-4-carboxylate

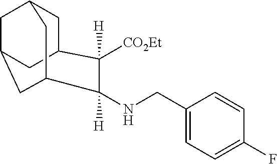

Thionyl chloride (0.36 mL, 4.93 mmol) was added dropwise to ethanol (6 mL) at 0° C. The solution was stirred at room temperature for 10 min, before it was added slowly to rac-(2S,5R)-3-Azatetracyclo[6.3.1.1^{6,10}.0^{2,5}]tridecan-4-one (prepared as described in Adv. Synth. Catal. 2004, 346, 566-572, 0.29 g, 1.50 mmol). The reaction was stirred at room temperature overnight. The mixture was concentrated and used directly for next step.

The crude was dissolved in ethanol (5 mL), followed by the sequential addition of 4-fluoro-benzaldehyde (0.16 mL, 1.50 mmol), sodium acetate (0.25 g, 3.00 mmol), sodium cyanoborohydride (0.19 g, 3.00 mmol) and 4 Å molecular sieves (0.4 g). The reaction was stirred at room temperature overnight before it was quenched with saturated aqueous sodium bicarbonate solution (10 mL). The mixture was stirred for additional 10 min. The mixture was extracted with ethyl acetate (10 mL×3). The organics were combined, dried over sodium sulfate, filtered and concentrated. Purification by flash column chromatography (Merck silica gel 60, 40-63 μm, 0-20% ethyl acetate in hexanes) afforded the desired product, rac-ethyl (4R,5S)-5-{[(4-fluorophenyl)methyl]amino}tricyclo[4.3.1.1^{3,8}]undecane-4-carboxylate (0.42 g, 1.23 mmol, 82%), as a clear oil. LC-MS (ESI) calcd for C₂₁H₂₈FNO₂345.45, found 346.4 [M+H⁺].

### (b) rac-N-{3-[(2S,7R)-3-[(4-Fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,2,4-benzothiadiazin-7-yl}methanesulfonamide

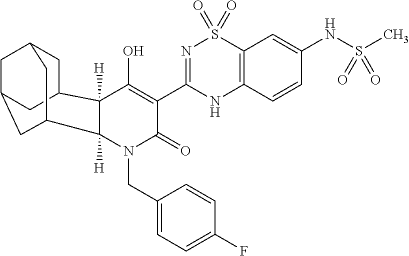

(7-Methanesulfonylamino-1,1-dioxo-1,4-dihydro-1λ⁶-benzo[1,2,4]thiadiazin-3-yl)-acetic acid (prepared as described in US 2010/0034773A1) (137 mg, 0.41 mmol) was dissolved in N,N-dimethylformamide (5 mL). rac-Ethyl (4R,5S)-5-{[(4-fluorophenyl)methyl]amino}tricyclo[4.3.1.1^{3,8}]undecane-4-carboxylate (143 mg, 0.41 mmol) was added followed by the addition of N-(3-dimethylaminopropyl)-N′-ethylcarbodiimide hydrochloride (196 mg, 1.02 mmol). The reaction was stirred at room temperature for 1 h. Triethylamine (0.68 mL, 4.92 mmol) was added and the mixture was stirred at 60° C. overnight. Upon cooling, the mixture was poured into 1.0 M aqueous hydrochloric acid solution (5 mL). The mixture was extracted with ethyl acetate (10 mL×3). The organics were combined, dried over sodium sulfate, filtered and concentrated. Purification by flash column chromatography (Merck silica gel 60, 40-63 μm, 0-80% ethyl acetate in hexanes) afforded the desired product, rac-N-{3-[(2S,7R)-3-[(4-fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,2,4-benzothiadiazin-7-yl}methanesulfonamide (160 mg, 0.26 mmol, 63%), as a white powder. ¹H NMR (400 MHz, Acetone-d₆) δ: 0.74-2.89 (15H, m), 3.09 (3H, s), 3.94-3.96 (1H, m), 4.21-4.24 (1H, m), 5.28-5.32 (1H, m), 7.10-7.80 (7H, m), 14.43 (1H, s). LC-MS (ESI) calcd for C₂₉H₃₁FN₄O₆S₂614.71, found 615.4 [M+H⁺].

### Example 6

### rac-N-({3-[(2S,7R)-3-[(4-Fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,5,2,4-thieno[2,3-e][1λ⁶,2,4]thiadiazin-7-yl}methyl)methanesulfonamide

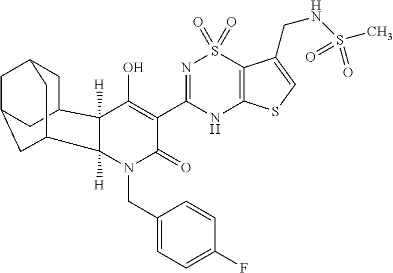

[7-(Methanesulfonylamino-methyl)-1,1-dioxo-1,4-dihydro-1λ⁶-thieno[2,3-e][1,2,4]thiadiazin-3-yl]-acetic acid (prepared as described in US 2009/0306057A1) (141 mg, 0.40 mmol), was dissolved in N,N-dimethylformamide (5 mL). rac-Ethyl (4R,5S)-5-{[(4-fluorophenyl)methyl]amino}tricyclo[4.3.1.1^{3,8}]undecane-4-carboxylate (prepared as described in Example 5, 138 mg, 0.40 mmol) was added followed by the addition of N-(3-dimethylaminopropyl)-N′-ethylcarbodiimide hydrochloride (192 mg, 1.00 mmol). The reaction was stirred at room temperature for 1 h. Triethylamine (0.67 mL, 4.80 mmol) was added and the mixture was stirred at 60° C. overnight. Upon cooling, the mixture was poured into 1.0 M aqueous hydrochloric acid solution (5 mL). The mixture was extracted with ethyl acetate (10 mL×3). The organics were combined, dried over sodium sulfate, filtered and concentrated. Purification by flash column chromatography (Merck silica gel 60, 40-63 μm, 0-80% ethyl acetate in hexanes) afforded the desired product, rac-N-({3-[(2S,7R)-3-[(4-fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,5,2,4-thieno[2,3-e][1λ⁶,2,4]thiadiazin-7-yl}methyl)methanesulfonamide (52 mg, 0.08 mmol, 20%), as a white powder. ¹H NMR (400 MHz, Acetone-d₆) δ: 0.66-3.27 (15H, m), 2.94 (3H, s), 4.18-4.50 (3H, m), 5.21-5.30 (1H, m), 7.00-7.48 (5H, m). LC-MS (ESI) calcd for C₂₈H₃₁FN₄O₆S₃ 634.76, found 635.2 [M+H⁺]. [7-(Methanesulfonylamino-methyl)-1,1-dioxo-1,4-dihydro-1λ⁶-thieno[2,3-e][1,2,4]thiadiazin-3-yl]-acetic acid (prepared as described in US 2009/0306057A1) (141 mg, 0.40 mmol), was dissolved in N,N-dimethylformamide (5 mL). rac-Ethyl (4R,5S)-5-{[(4-fluorophenyl)methyl]amino}tricyclo[4.3.1.1^{3,8}]undecane-4-carboxylate (prepared as described in Example 5, 138 mg, 0.40 mmol) was added followed by the addition of N-(3-dimethylaminopropyl)-N′-ethylcarbodiimide hydrochloride (192 mg, 1.00 mmol). The reaction was stirred at room temperature for 1 h. Triethylamine (0.67 mL, 4.80 mmol) was added and the mixture was stirred at 60° C. overnight. Upon cooling, the mixture was poured into 1.0 M aqueous hydrochloric acid solution (5 mL). The mixture was extracted with ethyl acetate (10 mL×3). The organics were combined, dried over sodium sulfate, filtered and concentrated. Purification by flash column chromatography (Merck silica gel 60, 40-63 μm, 0-80% ethyl acetate in hexanes) afforded the desired product, rac-N-({3-[(2S,7R)-3-[(4-fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,5,2,4-thieno[2,3-e][1λ⁶,2,4]thiadiazin-7-yl}methyl)methanesulfonamide (52 mg, 0.08 mmol, 20%), as a white powder. ¹H NMR (400 MHz, Acetone-d₆) δ: 0.66-3.27 (15H, m), 2.94 (3H, s), 4.18-4.50 (3H, m), 5.21-5.30 (1H, m), 7.00-7.48 (5H, m). LC-MS (ESI) calcd for C₂₈H₃₁FN₄O₆S₃ 634.76, found 635.2 [M+H⁺].

### Example 7

### rac-N-{3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide

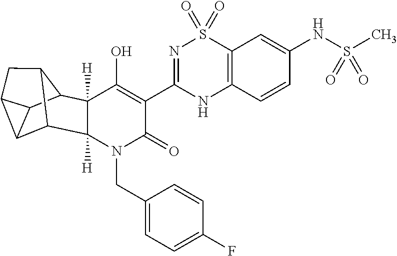

### (a) rac-(3S,6R)-4-Azapentacyclo[5.4.0.0^{2,9}.0^{3,6}.0^{8,10}]undecan-5-one

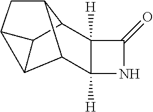

Tetracyclo[4.3.0.0^{2,4}.0^{3,7}]non-8-ene (prepared as described in J. Am. Chem. Soc. 1995, 117, 10276-10291, 30 mg, 0.25 mmol) was dissolved in chlorosulfonyl isocyanate (1 mL) and dichloromethane (0.5 mL). The reaction was stirred at 40° C. for 48 h. Upon cooling to room temperature, the reaction was added slowly to a solution of sodium sulfite (1.32 g), sodium phosphate dibasic (1.48 g), water (6.37 mL) and chloroform (5.28 mL). The mixture was diluted with water (10 mL) and extracted with ethyl acetate (10 mL×3). The organics were combined, dried over sodium sulfate, filtered and concentrated. Purification by flash column chromatography (Merck silica gel 60, 40-63 μm, 0-80% ethyl acetate in hexanes) afforded the desired product, rac-(3S,6R)-4-azapentacyclo[5.4.0.0^{2,9}.0^{3,6}.0^{8,10}]undecan-5-one (17 mg, 0.10 mmol, 40%), as a white solid. LC-MS (ESI) calcd for C₁₀H₁₁NO 161.20, found 162.2 [M+H⁺].

### rac-Ethyl (8R,9S)-9-{[(4-fluorophenyl)methyl]amino}tetracyclo[4.3.0.0^{2,4}.0^{3,7}]nonane-8-carboxylate

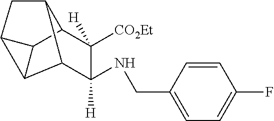

Thionyl chloride (0.12 mL, 1.64 mmol) was added dropwise to ethanol (2 mL) at 0° C. The solution was stirred at room temperature for 10 min, before it was added slowly to rac-(3S,6R)-4-azapentacyclo[5.4.0.0^{2,9}.0^{3,6}.0^{8,10}]undecan-5-one (17 mg, 0.10 mmol). The reaction was stirred at room temperature overnight. The mixture was concentrated and used directly for next step. The crude was dissolved in ethanol (2 mL), followed by the sequential addition of 4-fluoro-benzaldehyde (0.01 mL, 0.10 mmol), sodium acetate (17 mg, 0.20 mmol), sodium cyanoborohydride (13 mg, 0.20 mmol) and 4 Å molecular sieves (10 mg). The reaction was stirred at room temperature overnight before it was quenched with saturated aqueous sodium bicarbonate solution (10 mL). The mixture was stirred for additional 10 min. The mixture was extracted with ethyl acetate (10 mL×3). The organics were combined, dried over sodium sulfate, filtered and concentrated. Purification by flash column chromatography (Merck silica gel 60, 40-63 μm, 0-20% ethyl acetate in hexanes) afforded the desired product, rac-ethyl (8R,9S)-9-{[(4-fluorophenyl)methyl]amino}tetracyclo[4.3.0.0^{2,4}.0^{3,7}]nonane-8-carboxylate (30 mg, 0.10 mmol, 100%), as a clear oil. LC-MS (ESI) calcd for C₁₉H₂₂FNO₂ 315.38, found 316.2 [M+H⁺].

### (c) rac-N-{3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide

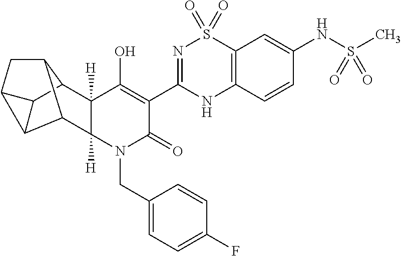

(7-Methanesulfonylamino-1,1-dioxo-1,4-dihydro-1λ⁶-benzo[1,2,4]thiadiazin-3-yl)-acetic acid (prepared as described in US 2010/0034773A1) (43 mg, 0.13 mmol), was dissolved in N,N-dimethylformamide (1 mL). rac-Ethyl (8R,9S)-9-{[(4-fluorophenyl)methyl]amino}tetracyclo[4.3.0.0^{2,4}.0^{3,7}]nonane-8-carboxylate (40 mg, 0.13 mmol) was added followed by the addition of N-(3-dimethylaminopropyl)-N′-ethylcarbodiimide hydrochloride (61 mg, 0.32 mmol) and 4-dimethylaminopyridine (2 mg, 0.01 mmol). The reaction was stirred at room temperature overnight. The mixture was poured into 1.0 M aqueous hydrochloric acid solution (2 mL). The mixture was extracted with ethyl acetate (4 mL×3). The organics were combined, dried over sodium sulfate, filtered and concentrated. The crude was dissolved in ethanol (1 mL). Sodium ethoxide (21 wt % in EtOH, 0.15 mL, 0.39 mmol) was added and the reaction was stirred at 60° C. overnight. The mixture was poured into 1.0 M aqueous hydrochloric acid solution (2 mL). The mixture was extracted with ethyl acetate (4 mL×3). The organics were combined, dried over sodium sulfate, filtered and concentrated. Purification by flash column chromatography (Merck silica gel 60, 40-63 μm, 0-80% ethyl acetate in hexanes) afforded the desired product rac-N-{3-[(1R,9S)-10-[(4-fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide (52 mg, 0.09 mmol, 69%), as a white powder. ¹H NMR (400 MHz, DMSO-d₆) δ: 1.02-3.45 (9H, m), 3.06 (3H, s), 3.84-3.96 (1H, m), 4.32-4.47 (1H, m), 4.93-5.06 (1H, m), 7.15-7.64 (7H, m), 10.20 (1H, s). LC-MS (ESI) calcd for C₂₇H₂₅FN₄O₆S₂ 584.64, found 585.4 [M+H⁺].

### Example 8

### rac-N-{3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide

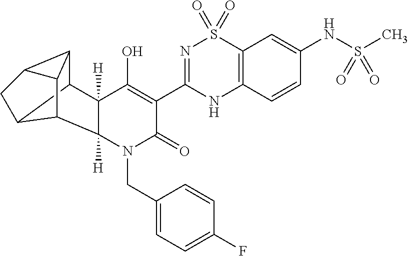

### (a) rac-Methyl (8R,9S)-9-{[(benzyloxy)carbonyl]amino}tetracyclo[4.3.0.0^{2,4}.0^{3,7}]nonane-8-carboxylate

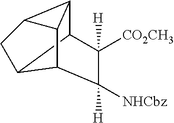

5-Oxapentacyclo[6.4.0.0^{2,10}.0^{3,7}.0^{9,11}]dodecane-4,6-dione (prepared as described in J. Chem. Soc. 1964, 5416-5421, 0.49 g, 2.56 mmol) was dissolved in triethylamine (1 mL) and methanol (10 mL). The reaction was stirred at room temperature overnight. The mixture was concentrated and used directly for next step. The crude was dissolved in tetrahydrofuran (10 mL) and cooled to 0° C. Triethylamine (1.07 mL, 7.68 mmol) was added followed by the dropwise addition of ethyl chloroformate (0.49 mL, 5.12 mmol). The reaction was stirred at 0° C. for 1 h. A solution of sodium azide (0.67 g, 10.24 mmol) in water (4 mL) was added to the reaction at 0° C. The reaction was stirred at 0° C. for 5 min, then at room temperature for 2 h. The mixture was diluted with water (5 mL) and half saturated sodium bicarbonate solution (5 mL). The mixture was extracted with ethyl acetate (10 mL×3). The organics were combined, dried over sodium sulfate, filtered, and concentrated to afford a clear oil. The oil was dissolved in benzene (8 mL) and refluxed for 2 h. Upon cooling to room temperature the solution was concentrated and dissolved in dichloromethane (8 mL). Benzyl alcohol (0.29 mL, 2.82 mmol) was added followed by triethylamine (0.71 mL, 5.12 mmol). The reaction was refluxed overnight. Upon cooling to room temperature the mixture was concentrated to afford a golden oil. Purification by flash column chromatography (Merck silica gel 60, 40-63 μm, 0-15% ethyl acetate in hexanes) afforded the desired product, rac-methyl (8R,9S)-9-{[(benzyloxy)carbonyl]amino}tetracyclo[4.3.0.0^{2,4}.0^{3,7}]nonane-8-carboxylate (0.71 g, 2.17 mmol, 85%), as a clear oil. LC-MS (ESI) calcd for C₁₉H₂₁Na₄ 327.37, found 328.4 [M+H⁺].

### (b) rac-Methyl (8R,9S)-9-{[(4-fluorophenyl)methyl]amino}tetracyclo[4.3.0.0^{2,4}.0^{3,7}]nonane-8-carboxylate

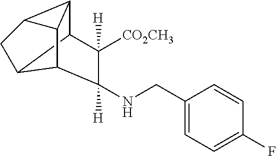

rac-Methyl (8R,9S)-9-{[(benzyloxy)carbonyl]amino}tetracyclo[4.3.0.0^{2,4}.0^{3,7}]nonane-8-carboxylate (0.71 g, 2.17 mmol) was dissolved in ethyl acetate (10 mL), followed by the addition of 10% palladium on carbon (0.1 g). The reaction was purged with hydrogen and was stirred under hydrogen atmosphere overnight. The mixture was filtered through a pad of Celite and concentrated. The crude was dissolved in methanol (6 mL), followed by the sequential addition of 4-fluoro-benzaldehyde (0.23 mL, 2.17 mmol), acetic acid (0.25 mL, 4.34 mmol) and sodium cyanoborohydride (0.27 g, 4.34 mmol). The reaction was stirred at 60° C. overnight. The reaction was cooled to room temperature, diluted with saturated aqueous sodium bicarbonate solution (20 mL) and stirred for additional 10 min. The mixture was extracted with ethyl acetate (20 mL×3). The organics were combined, dried over sodium sulfate, filtered and concentrated. Purification by flash column chromatography (Merck silica gel 60, 40-63 μm, 0-20% ethyl acetate in hexanes) afforded the desired product, rac-Methyl (8R,9S)-9-{[(4-fluorophenyl)methyl]amino}tetracyclo[4.3.0.0^{2,4}.0^{3,7}]nonane-8-carboxylate (0.42 g, 1.39 mmol, 64%), as a clear oil. ¹H NMR (400 MHz, CDCl₃) δ: 1.12-3.67 (11H, m), 3.60 (3H, s), 4.38-4.54 (1H, m), 7.07-7.41 (4H, m). LC-MS (ESI) calcd for C₁₈H₂₀FNO₂ 301.36, found 302.2 [M+H⁺].

### (c) rac-N-{3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide

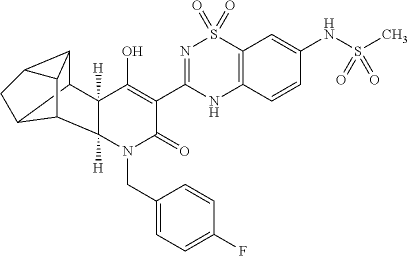

(7-Methanesulfonylamino-1,1-dioxo-1,4-dihydro-1λ⁶-benzo[1,2,4]thiadiazin-3-yl)-acetic acid (prepared as described in US 2010/0034773A1) (0.12 g, 0.36 mmol) was dissolved in N,N-dimethylformamide (1 mL). rac-Methyl (8R,9S)-9-{[(4-fluorophenyl)methyl]amino}tetracyclo[4.3.0.0^{2,4}.0^{3,7}]nonane-8-carboxylate (0.11 g, 0.36 mmol) was added followed by the addition of N-(3-dimethylaminopropyl)-N′-ethylcarbodiimide hydrochloride (0.17 g, 0.90 mmol) and 4-dimethylaminopyridine (5 mg, 0.04 mmol). The reaction was stirred at room temperature overnight. The mixture was poured into 1.0 M aqueous hydrochloric acid solution (5 mL). The mixture was extracted with ethyl acetate (10 mL×3). The organics were combined, dried over sodium sulfate, filtered and concentrated. The crude was dissolved in ethanol (1.5 mL). Sodium ethoxide (21 wt % in EtOH, 0.40 mL, 1.08 mmol) was added and the reaction was stirred at 60° C. overnight. The mixture was poured into 1.0 M aqueous hydrochloric acid solution (5 mL). The mixture was extracted with ethyl acetate (10 mL×3). The organics were combined, dried over sodium sulfate, filtered and concentrated. Purification by flash column chromatography (Merck silica gel 60, 40-63 μm, 0-80% ethyl acetate in hexanes) afforded the desired product, rac-N-{3-[(1R,9S)-10-[(4-fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide (0.14 g, 0.25 mmol, 69%), as a white powder. ¹H NMR (400 MHz, DMSO-d₆) δ: 0.83-3.49 (9H, m), 3.06 (3H, s), 3.95-4.02 (1H, m), 4.30-4.25 (1H, m), 5.10-5.14 (1H, m), 7.17-7.66 (7H, m), 10.20 (1H, s). LC-MS (ESI) calcd for C₂₇H₂₅FN₄O₆S₂ 584.64, found 585.4 [M+H⁺].

### Example 9

### rac-N-({3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,5,2,4-thieno[2,3-e][1λ⁶,2,4]thiadiazin-7-yl}methyl)methanesulfonamide

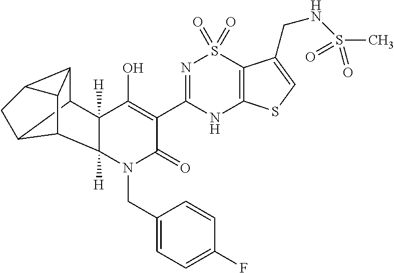

[7-(Methanesulfonylamino-methyl)-1,1-dioxo-1,4-dihydro-1λ⁶-thieno[2,3-e][1,2,4]thiadiazin-3-yl]-acetic acid (prepared as described in US 2009/0306057A1) 0.13 g, 0.36 mmol) was dissolved in N,N-dimethylformamide (1 mL). rac-Methyl (8R,9S)-9-{[(4-fluorophenyl)methyl]amino}tetracyclo[4.3.0.0^{2,4}.0^{3,7}]nonane-8-carboxylate (prepared as described in Example 8, 0.11 g, 0.36 mmol) was added followed by the addition of N-(3-dimethylaminopropyl)-N′-ethylcarbodiimide hydrochloride (0.17 g, 0.90 mmol) and 4-dimethylaminopyridine (5 mg, 0.04 mmol). The reaction was stirred at room temperature overnight. The mixture was poured into 1.0 M aqueous hydrochloric acid solution (5 mL). The mixture was extracted with ethyl acetate (10 mL×3). The organics were combined, dried over sodium sulfate, filtered and concentrated. The crude was dissolved in ethanol (1.5 mL). Sodium ethoxide (21 wt % in EtOH, 0.40 mL, 1.08 mmol) was added and the reaction was stirred at 60° C. overnight. The mixture was poured into 1.0 M aqueous hydrochloric acid solution (5 mL). The mixture was extracted with ethyl acetate (10 mL×3). The organics were combined, dried over sodium sulfate, filtered and concentrated. Purification by flash column chromatography (Merck silica gel 60, 40-63 μm, 0-80% ethyl acetate in hexanes) afforded the desired product, rac-N-({3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,5,2,4-thieno[2,3-e][1λ⁶,2,4]thiadiazin-7-yl}methyl)methanesulfonamide (0.15 g, 0.25 mmol, 69%), as a white powder. ¹H NMR (400 MHz, DMSO-d₆) δ: 0.86-3.41 (9H, m), 2.97 (3H, s), 4.36-4.16 (3H, m), 5.08-5.11 (1H, m), 7.15-7.80 (5H, m). LC-MS (ESI) calcd for C₂₆H₂₅FN₄O₆S₃ 604.69, found 605.0 [M+H⁺].

### Example 10

### rac-N-{3-[(1R,10S)-11-[(4-Fluorophenyl)methyl]-14-hydroxy-12-oxo-11-azahexacyclo[8.4.0.0^{2,70}.0^{3,5}.0^{4,9}.0^{6,8}]tetradec-13-en-13-yl]-1,1-dioxo-4H-1λ⁶,2,4-benzothiadiazin-7-yl}methanesulfonamide

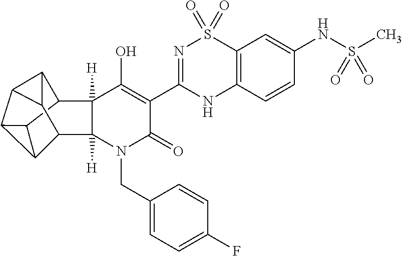

### (a) (1R,2R,6S,7R,8S,11R)-4-Oxatetracyclo[5.4.2.0^{2,6}.0^{8,11}]trideca-9,12-diene-3,5-dione

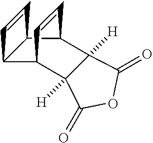

Following the procedure in J. Am. Chem. Soc. 1991, 113, p. 7882-7886, 1,3,5,7-cyclooctatetraene (25 g, 240 mmol) and maleic anhydride (25 g, 255 mmol) were combined in o-xylenes (35 mL). Hydroquinone (˜50 mg) was added and the mixture was heated in a sealed tube at 165° C. for 2 h. Upon cooling, the resulting precipitate was collected by vacuum filtration, rinsed with 1:1 diethyl ether/hexanes (˜50 mL) and recrystallized from acetone to afford (1R,2R,6S,7R,8S,11R)-4-oxatetracyclo[5.4.2.0^{2,6}.0^{8,11}]trideca-9,12-diene-3,5-dione (31.57 g, 156.1 mmol, 65% yield) as a white solid. ¹H NMR (400 MHz, CDCl₃) δ: 2.81-2.83 (2H, m), 3.07-3.09 (2H, m), 3.23-3.28 (2H, m), 5.91 (2H, s), 6.01-6.06 (2H, m).

### (b) 6-oxahexacyclo[7.4.0.0^{2,12}.0^{3,11}.0^{4,8}.0^{10,13}]tridecane-5,7-dione

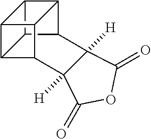

The synthesis of this compound was originally described in J. Am. Chem. Soc. 1991, 113, p. 7882-7886, and J. Am. Chem. Soc. 1971, 93, p. 2459-2463. (1R,2R,6S,7R,8S,11R)-4-oxatetracyclo[5.4.2.0^{2,6}.0^{8,11}]trideca-9,12-diene-3,5-dione (11 g, 54.4 mmol) was dissolved in acetone (500 mL). The solution was irradiated under N₂ at 25° C. with a 400-W Hanovia mercury lamp through a vicor filter for 10 h. Upon cooling the solution was concentrated in vacuo. The resulting solid was purified by flash column chromatography (ISCO, Superflash cartridge, gradient elution: 0→30% ethyl acetate in hexanes) followed by recrystallization from carbon tetrachloride to afford 6-oxahexacyclo[7.4.0.0^{2,12}.0^{3,11}.0^{4,8}.0^{10,13}]tridecane-5,7-dione (5.12 g, 25.3 mmol, 47% yield) as a white solid. ¹H NMR (400 MHz, CDCl₃) δ: 3.06-3.12 (4H, m), 3.20-3.25 (2H, m), 3.29-3.32 (2H, m), 3.33-3.36 (2H, m).

### (c) 6-Oxahexacyclo[7.4.0.0^{2,13}.0^{3,11}.0^{4,8}.0^{10,12}]tridecane-5,7-dione

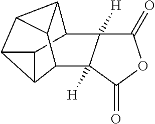

6-Oxahexacyclo[7.4.0.0^{2,12}.0^{3,11}.0^{4,8}.0^{10,13}]tridecane-5,7-dione (1.8 g, 8.9 mmol) was dissolved in acetone (50 mL). Silver tetrafluoroborate (1 g) was added and the reaction mixture was stirred at reflux under N₂ for 3 h. Additional silver tetrafluoroborate (1 g) was added and the mixture continued to stir at reflux under N₂ for 3 h. Upon cooling, the entire mixture was passed through a short plug of silica gel and the filtrate was concentrated in vacuo. The resulting residue was dissolved in ethyl acetate and passed through a short plug of silica gel, eluting with 1:1 hexanes/ethyl acetate. The filtrate was concentrated in vacuo. The resulting solid was purified by flash column chromatography (ISCO, Superflash cartridge, gradient elution: 0→30% ethyl acetate in hexanes) to afford 6-oxahexacyclo[7.4.0.0^{2,13}.0^{3,11}.0^{4,8}.0^{10,12}]tridecane-5,7-dione (1.75 g, 8.65 mmol, 97% yield) as a white solid. ¹H NMR (400 MHz, CDCl₃) δ: 1.50-1.54 (2H, m), 1.64-1.67 (2H, m), 1.94 (1H, sextet, J=3.3 Hz), 2.00 (1H, sextet, J=3.1 Hz), 3.00-3.03 (2H, m), 3.21-3.22 (2H, m).

### (d) rac-Methyl (9R,10S)-10-{[(benzyloxy)carbonyl]amino)pentacyclo[4.4.0.0^{2,4}.0^{3,8}.0^{5,7}]decane-9-carboxylate

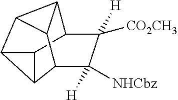

6-oxahexacyclo[7.4.0.0^{2,12}.0^{3,11}.0^{4,8}.0^{10,13}]tridecane-5,7-dione (0.4 g, 1.98 mmol) was dissolved in methyl alcohol (8 mL), triethyl amine (0.28 mL, 1.98 mmol) was added and the reaction mixture stirred at 25° C. for 16 h. The mixture was concentrated in vacuo. The residue was dissolved in benzene (20 mL) and concentrated in vacuo. The residue was dissolved in anhydrous tetrahydrofuran (10 mL). The flask was degassed and backfilled with nitrogen and the mixture was cooled to 0° C. Triethylamine (0.56 mL, 4 mmol) was added followed by the dropwise addition of ethyl chloroformate (0.38 mL, 4 mmol) with vigorous stirring. Immediate precipitation was observed. The mixture was stirred at 0° C. for 90 min. Sodium azide (0.39 g, 6 mmol) was dissolved in water (3 mL) and added to the reaction mixture at 0° C. The mixture was stirred at 0° C. for 10 min. The ice bath was removed. The mixture was warmed to 25° C. and was stirred for 1 h. The mixture was diluted with ethyl acetate (50 mL), washed with water (25 mL), aqueous sodium bicarbonate solution (25 mL), saturated aqueous brine solution (25 mL), dried over magnesium sulfate, filtered, and concentrated in vacuo. The resulting oil was dissolved in anhydrous benzene (20 mL) and concentrated in vacuo. The resulting oil was dissolved in anhydrous benzene (50 mL) and refluxed while stirring under nitrogen for 3 h. Upon cooling to 25° C. the solution was concentrated in vacuo. The resulting oil was dissolved in dichloromethane (30 mL) and benzyl alcohol (0.228 mL, 2.2 mmol) was added followed by triethylamine (0.56 mL, 4 mmol). The mixture stirred at 40° C. for 16 h. Upon cooling to 25° C. the solution was diluted with dichloromethane (50 mL), washed with 1.0 M aqueous hydrochloric acid solution (25 mL), saturated aqueous brine solution (25 mL), dried over magnesium sulfate, filtered, and concentrated in vacuo. Purification by flash column chromatography (ISCO, Superflash cartridge, gradient elution: 0→50% ethyl acetate in hexanes) afforded rac-methyl (9R,10S)-10-{[(benzyloxy)carbonyl]amino}pentacyclo[4.4.0.0^{2,4}.0^{3,8}.0^{5,7}]decane-9-carboxylate (0.49 g, 1.44 mmol, 73%) as a clear oil. LC-MS (ESI) calcd for C₂₀H₂₁Na₄ 339.15, found 340.2 [M+H⁺].

### (e) rac-Methyl (9R,10S)-10-aminopentacyclo[4.4.0.0^{2,4}.0^{3,8}.0^{5,7}]decane-9-carboxylate hydrochloride

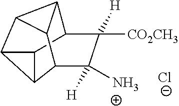

rac-Methyl (9R,10S)-10-{[(benzyloxy)carbonyl]amino}pentacyclo[4.4.0.0^{2,4}.0^{3,8}.0^{5,7}]decane-9-carboxylate (0.49 g, 1.44 mmol) was dissolved in ethyl acetate (20 mL) and methyl alcohol (20 mL). 10% Palladium on carbon (0.08 g) was added. The flask was degassed and backfilled with hydrogen gas via balloon. The mixture was stirred at 25° C. for 16 h. The mixture was passed through a plug of Celite and the filtrate was concentrated in vacuo to afford an oil. The oil was dissolved in diethyl ether (8 mL) and a 4.0 M solution of hydrochloric acid in dioxane (4 mL, 16 mmol) was added dropwise. Upon standing, a precipitate formed. The precipitate was collected by vacuum filtration and dried in vacuo to afford rac-methyl (9R,10S)-10-aminopentacyclo[4.4.0.0^{2,4}.0^{3,8}.0^{5,7}]decane-9-carboxylate hydrochloride (0.112 g, 0.46 mmol, 32% yield) as a white powder. LC-MS (ESI) calcd for C₁₂H₁₅NO₂ (free amine) 205.11, found 206.2 [M+H⁺].

### (f) rac-Methyl (9R,10S)-10-{[(4-fluorophenyl)methyl]amino}pentacyclo[4.4.0.0^{2,4}.0^{3,8}.0^{5,7}]decane-9-carboxylate

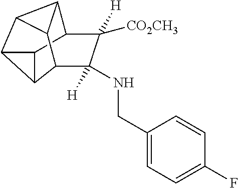

rac-Methyl (9R,10S)-10-aminopentacyclo[4.4.0.0^{2,4}.0^{3,8}.0^{5,7}]decane-9-carboxylate hydrochloride (0.102 g, 0.423 mmol) was dissolved in methyl alcohol (2.5 mL). Sodium acetate (0.082 g, 1 mmol) was added followed by 4-fluoro benzaldehyde (0.06 g, 0.487 mmol). Sodium cyanoborohydride (0.063 g, 1 mmol) was added and the mixture was shaken at 50° C. for 16 h. Upon cooling, saturated aqueous sodium bicarbonate solution (10 mL) was added and the mixture was shaken for 20 min. The resulting suspension was partitioned between ethyl acetate (40 mL) and saturated aqueous sodium bicarbonate solution (20 mL). The organic phase was further washed with saturated aqueous brine solution (20 mL), dried over magnesium sulfate, filtered, and concentrated in vacuo to afford crude rac-methyl (9R,10S)-10-{[(4-fluorophenyl)methyl]amino}pentacyclo[4.4.0.0^{2,4}.0^{3,8}.0^{5,7}]decane-9-carboxylate (0.13 g, 0.423 mmol) as a thick oil which was used directly in the next step without further purification. LC-MS (ESI) calcd for C₁₉H₂₀FNO₂ 313.15, found 314.4 [M+H⁺].

### (g) rac-N-{3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide

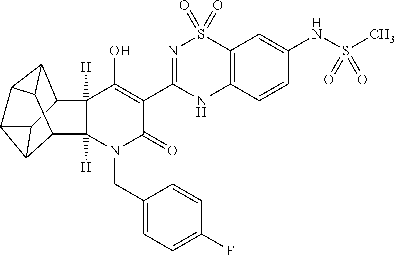

rac-Methyl (9R,10S)-10-{[(4-fluorophenyl)methyl]amino}pentacyclo[4.4.0.0^{2,4}.0^{3,8}.0^{5,7}]decane-9-carboxylate (0.066 g, 0.21 mmol) was dissolved in N,N-dimethylformamide (2 mL). (7-Methanesulfonylamino-1,1-dioxo-1,4-dihydro-1λ⁶-benzo[1,2,4]thiadiazin-3-yl)-acetic acid (prepared as described in U.S. Patent Application Publication No. US 2010/0034773A1) (0.074 g, 0.22 mmol) was added followed by 1-(3-dimethylaminopropyl)-3-ethylcarbodiimide hydrochloride (0.1 g, 0.525 mmol. The mixture stirred at 25° C. for 1 h. Triethylamine (0.5 mL, 3.57 mmol) was added and the mixture stirred at 60° C. for 16 h. Upon cooling to 25° C., the solution was diluted with ethyl acetate (50 mL), washed with 1.0 M aqueous hydrochloric acid solution (25 mL), saturated aqueous brine solution (25 mL), dried over magnesium sulfate, filtered, and concentrated in vacuo. Purification by flash column chromatography (ISCO, Superflash cartridge, gradient elution: 0→1% methyl alcohol in dichloromethane) afforded rac-N-{3-[(1R,10S)-11-[(4-fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide (0.038 g, 0.064 mmol, 30% yield) as a white powder. ¹H NMR (400 MHz, DMSO-d₆) δ: 1.24-1.30 (1H, m), 1.39-1.48 (2H, m), 1.56-1.61 (1H, m), 1.78-1.85 (2H, m), 2.53-2.56 (1H, m), 2.75-2.81 (1H, m), 2.96 (1H, d, J=3.0 Hz), 3.04 (3H, s), 3.92 (1H, d, J=11.0 Hz), 4.60 (1H, d, J=14.4 Hz), 4.92 (1H, d, J=15.6 Hz), 7.15 (2H, t, J=9.1 Hz), 7.39 (2H, dd, J₁=8.0 Hz, J₂=5.4 Hz), 7.50 (1H, dd, J₁=9.0 Hz, J₂=1.9 Hz), 7.56-7.61 (2H, m), 10.17 (1H, s). LC-MS (ESI) calcd for C₂₈H₂₅FN₄O₆S₂ 596.12, found 597.0 [M+H⁺].

### Example 11

### N-{3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide

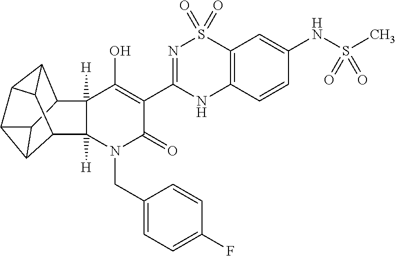

Both enantiomers of rac-N-{3-[(1R,10S)-11-[(4-fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide (0.028 g) were separated via chiral HPLC (Chiralpak AS-RH, 4.6×150 mm, 5 micron with mobile phases 20% 0.05% TFA in water and 80% 0.05% TFA in acetonitrile isocratic for 12 minutes, 0.7 mL/min, 310 nm, Rt=3.6 min) to afford pure N-{3-[(1S,10R)-11-[(4-fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide (0.011 g, 39%) as a white powder. LC-MS (ESI) calcd for C₂₈H₂₅FN₄O₆S₂ 596.12, found 597.0 [M+H⁺].

### Example 12

### N-{3-[(1S,10R)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl methanesulfonamide

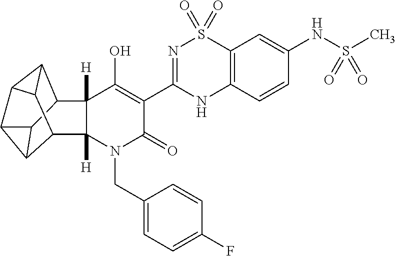

From the same chiral HPLC experiment described in Example 11 above, N-{3-[(1S,10R)-11-[(4-fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide (Rt=6.0 min) was isolated as a white powder (0.013 g, 46%). LC-MS (ESI) calcd for C₂₈H₂₅FN₄O₆S₂ 596.12, found 597.0 [M+H⁺].

### Example 13

### rac-N-({3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,5,2,4-thieno[2,3-e][1λ⁶,2,4]thiadiazin-7-yl}methyl)methanesulfonamide

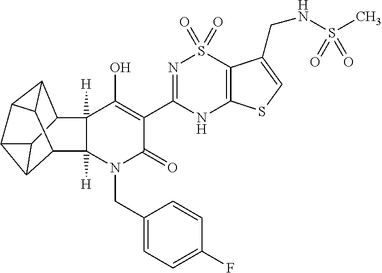

rac-Methyl (9R,10S)-10-{[(4-fluorophenyl)methyl]amino}pentacyclo[4.4.0.0^{2,4}.0^{3,8}.0^{5,7}]decane-9-carboxylate (prepared as described in Example 10) (0.066 g, 0.21 mmol, was dissolved in N,N-dimethylformamide (2 mL). [7-(Methanesulfonylamino-methyl)-1,1-dioxo-1,4-dihydro-1λ⁶-thieno[2,3-e][1,2,4]thiadiazin-3-yl]-acetic acid (prepared as described in U.S. Patent Application Publication No. US 2009/0306057A1) (0.074 g, 0.21 mmol) was added followed by 1-(3-dimethylaminopropyl)-3-ethylcarbodiimide hydrochloride (0.1 g, 0.525 mmol. The mixture stirred at 25° C. for 1 h. Triethylamine (0.5 mL, 3.57 mmol) was added and the mixture stirred at 60° C. for 16 h. The mixture stirred for an additional 5 h at 85° C. Upon cooling to 25° C., the solution was diluted with ethyl acetate (50 mL), washed with 1.0 M aqueous hydrochloric acid solution (25 mL), saturated aqueous brine solution (25 mL), dried over magnesium sulfate, filtered, and concentrated in vacuo. Purification by flash column chromatography (ISCO, Superflash cartridge, gradient elution: 0→1% methyl alcohol in dichloromethane) afforded rac-N-({3-[(1R,10S)-11-[(4-fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,5,2,4-thieno[2,3-e][1λ⁶,2,4]thiadiazin-7-yl}methyl)methanesulfonamide (0.0052 g, 0.008 mmol, 3.8% yield) as a white powder. LC-MS (ESI) calcd for C₂₇H₂₅FN₄O₆S₃ 616.09, found 617.2 [M+H⁺].

### Example 14

### rac-N-{3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,2,4-benzothiadiazin-7-yl}methanesulfonamide

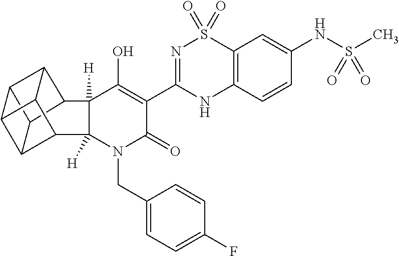

### (a) rac-Methyl (9R,10S)-10-aminopentacyclo[4.4.0.0^{2,5}.0^{3,8}.0^{4,7}]decane-9-carboxylate hydrochloride

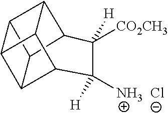

6-Oxahexacyclo[7.4.0.0^{2,12}.0^{3,11}.0^{4,8}.0^{10,13}]tridecane-5,7-dione (prepared as described above in Example 10) (0.5 g, 2.48 mmol), was dissolved in methyl alcohol (12 mL). Triethylamine (0.69 mL, 4.96 mmol) was added and the reaction mixture stirred at 25° C. for 16 h. The mixture was concentrated in vacuo. The residue was dissolved in benzene (20 mL) and concentrated in vacuo. The residue was dissolved in anhydrous tetrahydrofuran (20 mL). The flask was degassed and backfilled with nitrogen and the mixture was cooled to 0° C. Ethyl chloroformate (0.47 mL, 4.96 mmol) was added followed by triethylamine (0.35 mL, 2.48 mmol). Immediate precipitation was observed. The mixture was stirred at 0° C. for 2 h. Sodium azide (0.48 g, 7.4 mmol) was dissolved in water (3 mL) and added to the reaction mixture at 0° C. The mixture was stirred at 0° C. for 10 min. The ice bath was removed. The mixture was warmed to 25° C. and was stirred for 1 h. The mixture was diluted with ethyl acetate (50 mL), washed with aqueous sodium bicarbonate solution (25 mL), saturated aqueous brine solution (25 mL), dried over magnesium sulfate, filtered, and concentrated in vacuo. The resulting oil was dissolved in anhydrous benzene (20 mL) and concentrated in vacuo. The resulting oil was dissolved in anhydrous benzene (50 mL) and refluxed while stirring under nitrogen for 3 h. Upon cooling to 25° C. the solution was concentrated in vacuo. The resulting oil was dissolved in anhydrous tetrahydrofuran (20 mL) and added dropwise to 3.0 M aqueous hydrochloric acid solution while stirring at 0° C. The mixture continued to stir at 0° C. for 2 h. The mixture was concentrated in vacuo to afford crude rac-methyl (9R,10S)-10-aminopentacyclo[4.4.0.0^{2,5}.0^{3,8}.0^{4,7}]decane-9-carboxylate hydrochloride (0.35 g, 1.45 mmol, 58% yield) as a thick oil which was used directly in the next step without further purification. LC-MS (ESI) calcd for C₁₂H₁₅NO₂ 205.25, found 206.2 [M+H⁺].

### (b) rac-Methyl (9R,10S)-10-{[(4-fluorophenyl)methyl]amino}pentacyclo[4.4.0.0^{2,5}.0^{3,8}.0^{4,7}]decane-9-carboxylate

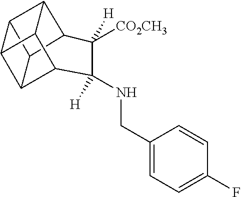

rac-Methyl (9R,10S)-10-aminopentacyclo[4.4.0.0^{2,5}.0^{3,8}.0^{4,7}]decane-9-carboxylate hydrochloride (0.35 g, 1.45 mmol) was dissolved in methyl alcohol (8 mL). Sodium acetate (0.24 g, 2.9 mmol) was added followed by 4-fluoro benzaldehyde (0.186 g, 1.5 mmol). The mixture stirred at 25° C. for 5 min. Sodium cyanoborohydride (0.27 g, 4.35 mmol) was added and the mixture stirred at 50° C. for 16 h. Upon cooling, saturated aqueous sodium bicarbonate solution (10 mL) was added and the mixture stirred for 20 min. The resulting suspension was partitioned between ethyl acetate (80 mL) and saturated aqueous sodium bicarbonate solution (40 mL). The organic phase was further washed with saturated aqueous brine solution (30 mL), dried over magnesium sulfate, filtered, and concentrated in vacuo. Purification by flash column chromatography (ISCO, Superflash cartridge, gradient elution: 0→50% ethyl acetate in hexanes) afforded rac-methyl (9R,10S)-10-{[(4-fluorophenyl)methyl]amino}pentacyclo[4.4.0.0^{2,5}.0^{3,8}.0^{4,7}]decane-9-carboxylate (0.16 g, 0.51 mmol, 35% yield) as an oil. LC-MS (ESI) calcd for C₁₉H₂₀FNO₂ 313.15, found 314.4 [M+H⁺].

### (c) rac-N-{3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,2,4-benzothiadiazin-7-yl}methanesulfonamide

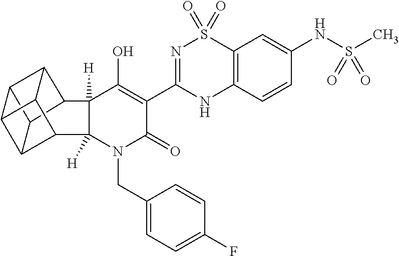

rac-Methyl (9R,10S)-10-{[(4-fluorophenyl)methyl]amino}pentacyclo[4.4.0.0^{2,5}.0^{3,8}.0^{4,7}]decane-9-carboxylate (0.08 g, 0.255 mmol) was dissolved in N,N-dimethylformamide (3 mL). (7-methanesulfonylamino-1,1-dioxo-1,4-dihydro-1λ⁶-benzo[1,2,4]thiadiazin-3-yl)-acetic acid (prepared as described in U.S. Patent Application Publication No. US 2010/0034773A1) (0.087 g, 0.26 mmol) was added followed by 1-(3-dimethylaminopropyl)-3-ethylcarbodiimide hydrochloride (0.15 g, 0.765 mmol. The mixture stirred at 25° C. for 1 h. The mixture was diluted with ethyl acetate (50 mL), washed with 1.0 M aqueous hydrochloric acid solution (25 mL), saturated aqueous brine solution (25 mL), dried over magnesium sulfate, filtered, and concentrated in vacuo. The residue was dissolved in N,N-dimethylformamide (3 mL). Triethylamine (0.5 mL, 3.57 mmol) was added and the mixture stirred at 75° C. for 5 h. Upon cooling to 25° C., the solution was diluted with ethyl acetate (50 mL), washed with 1.0 M aqueous hydrochloric acid solution (25 mL), saturated aqueous brine solution (25 mL), dried over magnesium sulfate, filtered, and concentrated in vacuo. Purification by flash column chromatography (ISCO, Superflash cartridge, gradient elution: 0→100% ethylacetate in hexanes) afforded rac-N-{3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,2,4-benzothiadiazin-7-yl}methanesulfonamide (0.018 g, 0.03 mmol, 12% yield) as a white powder. LC-MS (ESI) calcd for C₂₈H₂₅FN₄O₆S₂ 596.12, found 597.2 [M+H⁺].

### Example 15

### rac-N-({3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,5,2,4-thieno[2,3-e][1λ⁶,2,4]thiadiazin-7-yl}methyl)methanesulfonamide

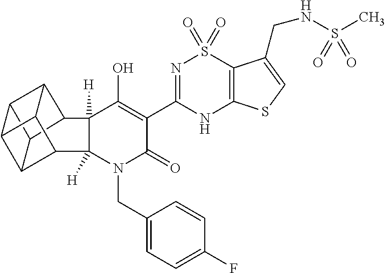

rac-Methyl (9R,10S)-10-{[(4-fluorophenyl)methyl]amino}pentacyclo[4.4.0.0^{2,5}.0^{3,8}.0^{4,7}]decane-9-carboxylate (prepared as described above in Example 14) (0.08 g, 0.255 mmol), was dissolved in N,N-dimethylformamide (3 mL). [7-(Methanesulfonylamino-methyl)-1,1-dioxo-1,4-dihydro-1λ⁶-thieno[2,3-e][1,2,4]thiadiazin-3-yl]-acetic acid (prepared as described in U.S. Patent Application Publication No. US 2009/0306057A1) (0.092 g, 0.26 mmol) was added followed by 1-(3-dimethylaminopropyl)-3-ethylcarbodiimide hydrochloride (0.15 g, 0.77 mmol. The mixture stirred at 25° C. for 1 h. The mixture was diluted with ethyl acetate (50 mL), washed with 1.0 M aqueous hydrochloric acid solution (25 mL), saturated aqueous brine solution (25 mL), dried over magnesium sulfate, filtered, and concentrated in vacuo. The residue was dissolved in N,N-dimethylformamide (3 mL). Triethylamine (0.5 mL, 3.57 mmol) was added and the mixture stirred at 75° C. for 18 h. Upon cooling to 25° C., the solution was diluted with ethyl acetate (50 mL), washed with 1.0 M aqueous hydrochloric acid solution (25 mL), saturated aqueous brine solution (25 mL), dried over magnesium sulfate, filtered, and concentrated in vacuo. Purification by flash column chromatography (ISCO, Superflash cartridge, gradient elution: 0→100% ethylacetate in hexanes) followed by preparative thin layer chromatography (75% ethylacetate in hexanes) afforded rac-N-({3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,5,2,4-thieno[2,3-e][1λ⁶,2,4]thiadiazin-7-yl}methyl)methanesulfonamide (0.0012 g, 0.002 mmol, 1% yield) as a white powder. LC-MS (ESI) calcd for C₂₇H₂₅FN₄O₆S₃ 616.09, found 617.2 [M+H⁺].

### Example 16

### rac-N-{3-[(4S,9R)-14,14-Difluoro-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methane sulfonamide

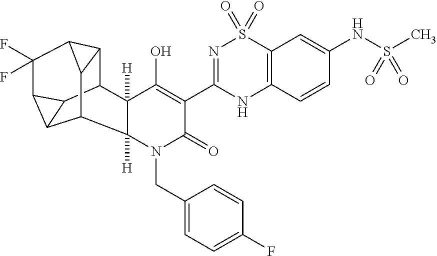

### (a) Bicyclo[4.2.1]nona-2,4,7-trien-9-one

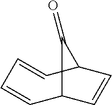

This compound was originally reported in J. Org. Chem. Soc. 1972, 37, p. 2517-2519 and J. Am. Chem. Soc. 1972, 94, p. 5366-5373. A flask containing metallic lithium granules (2.27 g, 327 mmol) was degassed while being gently warmed and was then back filled with He (g). Anhydrous diethyl ether (140 mL) was added. 1,3,5,7-Cyclooctatetraene (17 g, 163 mmol) was dissolved in anhydrous diethyl ether (20 mL) and filtered to remove a slight precipitate. The cyclooctatetraene/ether solution was added dropwise to the lithium/ether mixture while stirring vigorously at room temperature. The mixture continued to stir vigorously under He (g) at 25° C. for 5 h. Additional anhydrous diethyl ether (120 mL) was added and the mixture continued to stir vigorously under He (g) at 25° C. for 40 h. The resulting suspension was chilled to 0° C. under He (g). Dimethyl carbamoyl chloride (15.3 mL, 167 mmol) was dissolved in anhydrous diethyl ether (50 mL) and added to the di-anion solution while stirring vigorously under He (g) at 0° C. The mixture continued to stir vigorously under He (g) at 0° C. for 4 h. At 0° C., a 3M aqueous sulfuric acid solution (200 mL, 600 mmol) was slowly added to quench the reaction. The product was extracted into diethyl ether (2×200 mL) and concentrated in vacuo. Purification by flash column chromatography (ISCO, Superflash cartridge, gradient elution: 0→30% ethyl acetate in hexanes) afforded bicyclo[4.2.1]nona-2,4,7-trien-9-one (10.3 g, 78 mmol, 48% yield) as a clear oil. The product was stored in frozen benzene at −20° C. ¹H NMR (400 MHz, CDCl₃) δ: 3.10 (2H, d, J=8.7 Hz), 5.77-5.83 (4H, m), 5.86-5.91 (2H, m).

### (b) tricyclo[3.3.1.0^{2,8}]nona-3,6-dien-9-one

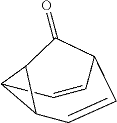

This compound was originally reported in J. Am. Chem. Soc. 1972, 94, p. 5366-5373. Bicyclo[4.2.1]nona-2,4,7-trien-9-one (8 g, 60.5 mmol) and 4,4′-bis(N,N-dimethylamino)benzophenone (16.8 g, 62.6 mmol, known as Michler's ketone) were dissolved in benzene. The solution was irradiated while bubbling N₂ through the mixture at 25° C. with a 400-W Hanovia mercury lamp through a Pyrex filter for 1 h. Upon cooling, the mixture was concentrated in vacuo. Purification by flash column chromatography (ISCO, Superflash cartridge, gradient elution: 0→30% ethyl acetate in hexanes) afforded tricyclo[3.3.1.0^{2,8}]nona-3,6-dien-9-one (3.2 g, 24 mmol, 40% yield) as a white solid. The product was stored at −20° C. ¹H NMR (400 MHz, Acetone-d₆) δ: 2.65 (2H, quintet, J=4.3 Hz), 4.38 (4H, bs), 5.74-5.79 (2H, m).

### (c) 6-Oxahexacyclo[7.5.0.0^{2,14}.0^{3,11}.0^{4,8}.0^{10,12}]tetradecane-5,7,13-trione

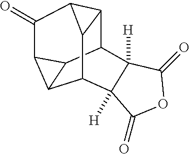

This compound was prepared using the procedure described Helvetica Chemica Acta, 1990, 73, p. 1182-1196. Tricyclo[3.3.1.0^{2,8}]nona-3,6-dien-9-one (2.5 g, 18.9 mmol) was suspended in o-xylenes (25 mL). Maleic anhydride (3.7 g, 37.9 mmol) was added followed by hydroquinone (˜25 mg). The mixture stirred at 155° C. for 60 h. Upon cooling, the resulting crystals were collected by vacuum filtration, rinsed with o-xylenes and dried in vacuo to afford 6-oxahexacyclo[7.5.0.0^{2,14}.0^{3,11}.0^{4,8}.0^{10,12}]tetradecane-5,7,13-trione (1.87 g, 8.1 mmol, 43% yield) as beige crystals. ¹H NMR (400 MHz, DMSO-d₆) δ: 1.32-1.37 (1H, m), 1.54-1.58 (1H, m), 1.73 (2H, dd, J₁=7.8 Hz, J₂=2.4 Hz), 1.98 (2H, dd, J₁=7.8 Hz, J₂=2.3 Hz), 3.01 (2H, s), 3.57 (2H, s).

### (d) rac-10-Benzyl-11-methyl (10S,11R)-5-oxopentacyclo[5.4.0.0^{2,4}.0^{3,9}.0^{6,8}]undecane-10,11-dicarboxylate

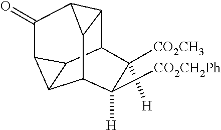

6-Oxahexacyclo[7.5.0.0^{2,14}.0^{3,11}.0^{4,8}.0^{10,12}]tetradecane-5,7,13-trione (1.5 g, 6.51 mmol) was suspended in methyl alcohol (30 mL). Triethylamine (1 mL, 7.16 mmol) was added and the mixture stirred at 25° C. for 16 h. The solution was concentrated in vacuo to afford a thick oil. The oil was dissolved in toluene and concentrated in vacuo. The resulting oil was dissolved in acetonitrile (30 mL). Triethylamine (1.36 mL, 9.77 mmol) was added followed by (2-(7-Aza-1H-benzotriazole-1-yl)-1,1,3,3-tetramethyluronium hexafluorophosphate) (2.96 g, 7.8 mmol) followed by benzyl alcohol (1.4 g, 13.02 mmol). The mixture stirred at 25° C. for 16 h. The solution was concentrated in vacuo to remove the solvent. The resulting oil was dissolved in ethyl acetate (120 mL), washed with saturated aqueous sodium bicarbonate solution (60 mL)), saturated aqueous brine solution (25 mL), dried over magnesium sulfate, filtered, and concentrated in vacuo. Purification by flash column chromatography (ISCO, Superflash cartridge, gradient elution: 0→100% ethyl acetate in hexanes) afforded rac-10-benzyl 11-methyl (10S,11R)-5-oxopentacyclo[5.4.0.0^{2,4}.0^{3,9}.0^{6,8}]undecane-10,11-dicarboxylate (1.9 g, 5.39 mmol, 83%) as a thick oil. LC-MS (ESI) calcd for C₂₁H₂₀O₅ 352.13, found 353.4 [M+H⁺]. ¹H NMR (400 MHz, DMSO-d₆) δ: 1.31-1.44 (2H, m), 1.61-1.77 (2H, m), 1.88-2.00 (2H, m), 2.44-2.55 (2H, m), 2.89-3.00 (2H, m), 3.25-3.38 (2H, m), 3.67 (3H, s), 5.17 (2H, quartet, J=11.3 Hz), 7.32-7.38 (5H, m).

### (e) rac-10′-methyl 11′-phenyl (10′R,11′S)-spiro[1,3-d]thiolane-2,5′-pentacyclo[5.4.0.0^{2,4}.0^{3,9}.0^{6,8}]undecane]-10′,11′-dicarboxylate

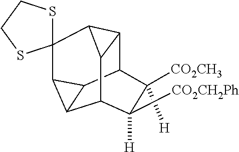

rac-10-Benzyl 11-methyl (10S,11R)-5-oxopentacyclo[5.4.0.0^{2,4}.0^{3,9}.0^{6,8}]undecane-10,11-dicarboxylate (1.5 g, 4.26 mmol) was dissolved in dichloromethane (100 mL) and chilled to 0° C. under N₂. Ethane-1,2-dithiol (0.45 mL, 5.33 mmol) was added followed by stannic chloride (0.2 mL, 1.7 mmol) and the mixture stirred at 0° C. for 20 min. Additional ethane-1,2-dithiol (0.45 mL, 5.33 mmol) was added followed by stannic chloride (0.3 mL, 2.55 mmol). The ice bath was removed and the mixture stirred at 25° C. for 70 min. Half saturated aqueous sodium bicarbonate solution (150 mL) was added and the mixture stirred for 5 min. The mixture was diluted with dichloromethane (450 mL), shaken and the organic phase was passed through a short plug of silica gel. The resulting solution was concentrated in vacuo to afford rac-10′-methyl 11′-phenyl (10′R,11′S)-spiro[1,3-d]thiolane-2,5′-pentacyclo[5.4.0.0^{2,4}.0^{3,9}.0^{6,8}]undecane]-10′,11′-dicarboxylate (1.48 g, 3.46 mmol, 81%) as a white solid. LC-MS (ESI) calcd for C₂₃H₂₄O₄S₂ 428.11, found 429.0 [M+H⁺].

### (f) rac-10-Benzyl-11-methyl (10S,11R)-5,5-difluoropentacyclo[5.4.0.0^{2,4}.0^{3,9}.0^{6,8}]undecane-10,11-dicarboxylate

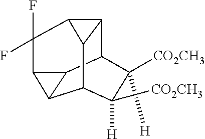

A solution of rac-10-benzyl-11-methyl (10S,11R)-5-oxopentacyclo[5.4.0.0^{2,4}.0^{3,9}.0^{6,8}]undecane-10,11-dicarboxylate in dichloromethane is prepared and cooled with an ice bath. Excess (diethylamino)sulfur trifluoride is added dropwise with stirring while raising the temperature to 20° C. The reaction is followed by TLC and LC-MS to observe consumption of the ketone starting material and conversion to the difluoro product. Upon completion the reaction solution is carefully neutralized with a saturated aqueous sodium bicarbonate solution and extracted with excess dichloromethane. The organic phase is then separated and dried over anhydrous magnesium sulfate, concentrated under vacuum and purified by column chromatography with silica to give rac-10-benzyl-11-methyl (10S,11R)-5,5-difluoropentacyclo[5.4.0.0^{2,4}.0^{3,9}.0^{6,8}]undecane-10,11-dicarboxylate.

### (g) rac-(10S,11R)-5,5-difluoropentacyclo[5.4.0.0^{2,4}.0^{3,9}.0^{6,8}]undecane-10,11-dicarboxylic acid 10-methyl ester

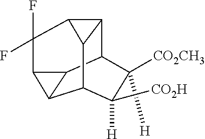

A solution of rac-10-benzyl-11-methyl (10S,11R)-5,5-difluoropentacyclo[5.4.0.0^{2,4}.0^{3,9}.0^{6,8}]undecane-10,11-dicarboxylate dissolved in ethyl acetate followed by the addition of a catalytic amount of 10% palladium on carbon, and purged with hydrogen is stirred under an hydrogen atmosphere overnight. The mixture is then filtered through a pad of celite and concentrated and the crude mono acid product is used directly for the next step.

### (h) rac-Methyl (10R,11S)-11-{[(benzyloxy)carbonyl]amino}5,5-difluoropentacyclo[5.4.0.0^{2,4}.0^{3,9}.0^{6,8}]undecane-10-carboxylate

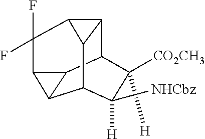

rac-(10S,11R)-5,5-Difluoropentacyclo[5.4.0.0^{2,4}.0^{3,9}.0^{6,8}]undecane-10,11-dicarboxylic acid 10-methyl ester is dissolved in tetrahydrofuran and cooled to 0° C. Excess triethylamine is added followed by the dropwise addition of ethyl chloroformate with stirring at 0° C. An aqueous solution of sodium azide is then added to the reaction mixture at 0° C. with stirring at 0° C. for 5 min, then at room temperature for 2 h converting the mixed anhydride to an acyl azide. Diluting the mixture with water and half saturated sodium bicarbonate solution, and extracting with ethyl acetate, drying the combined organic layers over sodium sulfate, filtering and concentrating provides the crude acyl azide which can further be converted to the isocynate by careful heating in benzene. Upon cooling to room temperature the solution is concentrated and dissolved in dichloromethane. Addition of benzyl alcohol and excess triethylamine and refluxing overnight followed by concentration under vacuum gives the crude benzyl carbamate which is normally purified by column chromatography on silica.

### (i & j) rac-Methyl (10R,11S)-11-{[(4-fluorophenyl)methyl]amino)-5,5-difluoropentacyclo[5.4.0.0^{2,4}.0^{3,9}.0^{6,8}]undecane-10-carboxylate

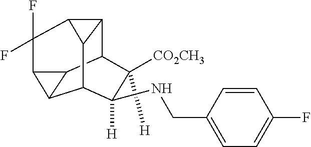

A solution of rac-Methyl (10R,11S)-11-{[(benzyloxy)carbonyl]amino}5,5-difluoropentacyclo[5.4.0.0^{2,4}.0^{3,9}.0^{6,8}]undecane-10-carboxylate in ethyl acetate with 10% palladium on carbon is purged with hydrogen and further stirred under hydrogen atmosphere overnight. Upon removal of the Cbz group, the mixture is then filtered through a pad of celite and concentrated. The crude amine can be used directly for the next step. The crude amine is dissolved in methanol, followed by the sequential addition of 4-fluoro-benzaldehyde, acetic acid, and sodium cyanoborohydride. The reaction mixture is then stirred at 60° C. for 12 h. The reaction mixture is then cooled to room temperature, diluted with saturated aqueous sodium bicarbonate solution and stirred. The mixture can then be extracted with ethyl acetate. Combining the organics layers, drying over sodium sulfate, filtration and concentration and purification by flash column chromatography on silica can yield the desired product.

### (k) rac-N-{3-[(4S,9R)-14,14-Difluoro-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide

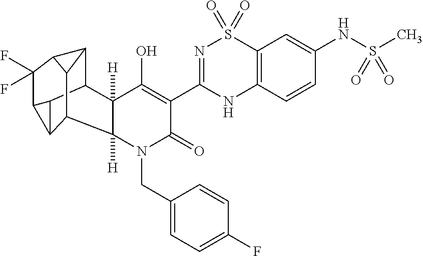

rac-Methyl (10R,11S)-11-{[(4-fluorophenyl)methyl]amino)-5,5-difluoropentacyclo[5.4.0.0^{2,4}.0^{3,9}.0^{6,8}]undecane-10-carboxylate dissolved in N,N-dimethylformamide is added (7-methanesulfonylamino-1,1-dioxo-1,4-dihydro-1λ⁶-benzo[1,2,4]thiadiazin-3-yl)-acetic acid (prepared as described in U.S. Patent Application Publication No. US 2010/0034773A1) followed by 1-(3-dimethylaminopropyl)-3-ethylcarbodiimide hydrochloride. The mixture is then stirred at 25° C. until the amine is consumed and the intermediate amide is detected by TLC and LC-MS. The mixture can then be diluted with ethyl acetate washed with 1.0 M aqueous hydrochloric acid solution, saturated aqueous brine solution, dried over magnesium sulfate, filtered, and concentrated in vacuo. The residue is then further dissolved in N,N-dimethylformamide whereupon triethylamine is added and the mixture stirred at 75° C. for 5 h or until LC-MS shows consumption of the intermediate amide and product formation. Upon cooling to 25° C., the solution is diluted with ethyl acetate, washed with 1.0 M aqueous hydrochloric acid solution, saturated aqueous brine solution, dried over magnesium sulfate, filtered, and concentrated in vacuo. Purification, typically by flash column chromatography (ISCO, Superflash cartridge, gradient elution: 0→100% ethylacetate in hexanes) affords the desired product.

### Example 17

### Reference Compound

### Bioorg. Med. Chem. Lett., 19, 6404 (2009)

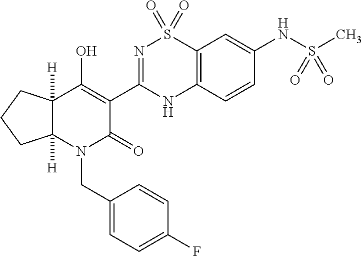

### Example 18

### Reference Compound

### Bioorg. Med. Chem. Lett., 19, 6404 (2009)

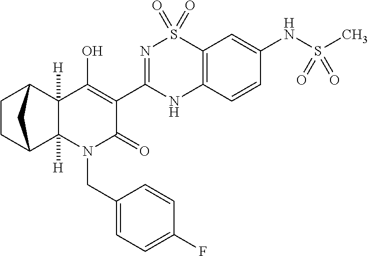

### Example 19

### Reference Compound

### U.S. Patent Publication No. 2009/0306057A1, pg. 48

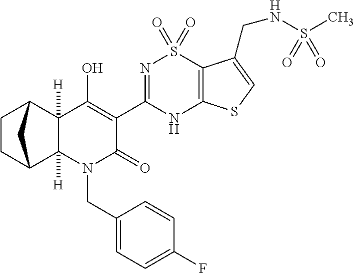

### Example 20

### Reference Compound

### Bioorg. Med. Chem. Lett., 19, 6404 (2009)

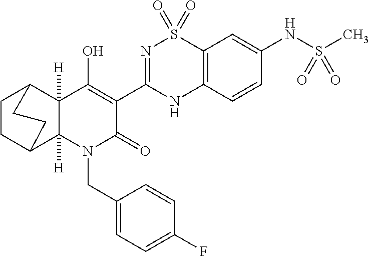

### Example 21

### Reference Compound

### Bioorg. Med. Chem. Lett., 18, 3616 (2008)

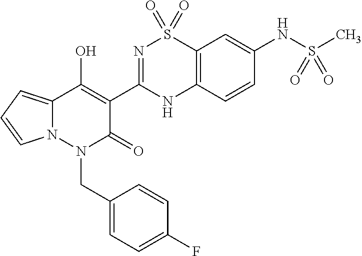

### Biological Testing

The ability of compounds of the invention to inhibit HCV replication can be demonstrated in the following in vitro assays.

### Transient Replicon Assay

The transient replicon assay utilizes the genotype 1b (Con1) dicistronic subgenomic replicon. Genotype 1a activity was determined using a chimeric replicon containing the genotype 1b sequence for NS3 through NS5A nonstructural proteins and genotype 1a (H77) sequence for the NS5B region.

The RNA transcripts were generated using the Megascript T7 kit (Applied Biosystems, Foster City, Calif.). Exponentially growing Huh7-Lunet cells were mixed with 4 μg of replicon RNA and plated into a 96-well plate Gene Pulser MXcell Electroporation System (Bio-Rad, Hercules, Calif.). Electroporation was immediately performed at 250 V and 750 μF capacitance with a single exponential waveform pulse. Transfected cells were transferred to Dulbecco modified Eagle medium in the presence of 10% FBS and plated into 96-well plates with 7,500 cells per well. Compounds at various concentrations were added to the cells after 2 hours and were cultured for 4 days. The cells were lysed with the Bright-Glo Reagent (Promega, Madison, Wis.) and luciferase activity was measured with a luminometer (Wallac 1420 Multilabel HTS Counter Victor 2). The background control was replicon cells treated with 100 nM BILN-2061, an inhibitor of the HCV protease. Dose responses were performed in triplicate and the values were averaged. All experiments were repeated at least three times to verify the reproducibility.

The EC₅₀ values were determined using a standard four-parameter dose response equation.

| Example # | EC₅₀ 1b (nM) | EC₅₀ 1a (nM) |
|---|---|---|
| 1 | 2 | 12 |
| 2 | 1 | 8 |
| 3 | >330 | >330 |
| 4 | 1 | 42 |
| 5 | 11 | 193 |
| 6 | 42 | 696 |
| 7 | 4 | 137 |
| 8 | 4 | 336 |
| 9 | 5 | 171 |
| 10 | 1 | 8 |
| 11 | 0.5 | 4 |
| 12 | >330 | >330 |
| 13 | 2 | 36 |
| 14 | 2 | 43 |
| 15 | 13 | NA |
| 17 | 18 | 550 |
| 18 | 2 | 45 |
| 19 | 2 | 45 |
| 20 | 0.6 | 12 |
| 21 | 13 | 400 |

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention.

What is claimed is:

1. A compound of Formula I 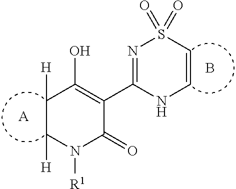
wherein
Ring A is selected from 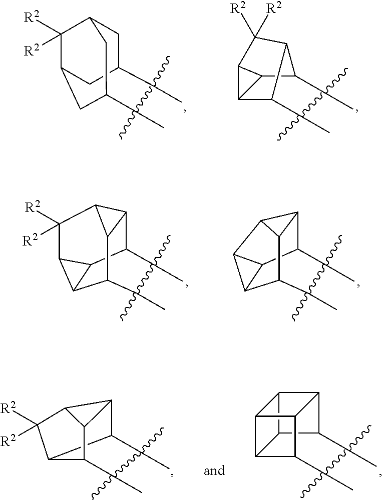
Ring B is 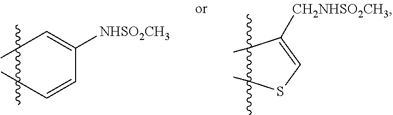
R¹ is alkyl or —C₁-C₆ alkylene(aryl),
R² is independently H, F, OH, OR³, —C₁-C₆ alkyl, C₃-C₈ cycloalkyl, —C₁-C₆ alkylene(C₃-C₈ cycloalkyl), —C₁-C₆ alkylene(aryl), —C₁-C₆ alkylene(heterocyclyl), aryl, or heterocyclyl, or both of the R² substituents are OCH₃, form an oxo, or form a ring comprised of —OCH₂CH₂O— or —SCH2CH₂S—, and
R³ is a tert-butyl or CH₂Ph,
wherein the above alkyl, alkylene, cycloalkyl, aryl, and heterocyclyl moieties are optionally and independently substituted by 1-4 substituents selected from hydrogen, alkylamine, amino, aryl, cycloalkyl, heterocyclyl, azido, C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, C₂-C₆ alkynyl, carboxyl, cyano, halo, hydroxyl, or nitro,
or a pharmaceutically acceptable salt or tautomer thereof.

2. The compound according to claim 1 wherein Ring A is

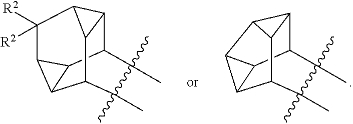

3. The compound according to claim 1 wherein Ring B is

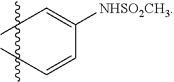

4. The compound according to claim 1 wherein R¹ is —CH₂-aryl.

5. The compound according to claim 1 wherein R² is H.

6. A compound selected from
rac-N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide;
N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide;
N-{3-[(4R,9S)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide;
rac-N-({3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,5,2,4-thieno[2,3-e][1λ⁶,2,4]thiadiazin-7-yl}methyl)methanesulfonamide;
rac-N-{3-[(2S,7R)-3-[(4-Fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,2,4-benzothiadiazin-7-yl}methanesulfonamide;
rac-N-({3-[(2S,7R)-3-[(4-Fluorophenyl)methyl]-6-hydroxy-4-oxo-3-azatetracyclo[8.3.1.1^{8,12}.0^{2,7}]pentadec-5-en-5-yl]-1,1-dioxo-4H-1λ⁶,5,2,4-thieno[2,3-e][1λ⁶,2,4]thiadiazin-7-yl}methyl)methanesulfonamide;
rac-N-{3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide;
rac-N-{3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,2,4-benzothiadiazin-7-yl}methanesulfonamide;
rac-N-({3-[(1R,9S)-10-[(4-Fluorophenyl)methyl]-13-hydroxy-11-oxo-10-azapentacyclo[7.4.0.0^{2,7}.0^{3,5}.0^{4,8}]tridec-12-en-12-yl]-1,1-dioxo-4H-1λ⁶,5,2,4-thieno[2,3-e][1λ⁶,2,4]thiadiazin-7-yl}methyl)methanesulfonamide;
rac-N-{3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide;
N-{3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide;
N-{3-[(1S,10R)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl methanesulfonamide;
rac-N-({3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,5,2,4-thieno[2,3-e][1λ⁶,2,4]thiadiazin-7-yl}methyl)methanesulfonamide;
rac-N-{3-[(1R10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,2,4-benzothiadiazin-7-yl}methanesulfonamide;
rac-N-({3-[(1R,10S)-11-[(4-fluorophenyl)methyl]-14-hydroxy-12-oxo-11-azahexacyclo[8.4.0.0^{2,5}.0^{3,8}.0^{4,7}.0^{6,9}]tetradec-13-en-13-yl]-1,1-dioxo-4H-1λ⁶,5,2,4-thieno[2,3-e][1λ⁶,2,4]thiadiazin-7-yl}methyl)methanesulfonamide; and
rac-N-{3-[(4S,9R)-14,14-difluoro-5-[(4-fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide,
or a pharmaceutically acceptable salt or tautomer thereof.

7. The compound according to claim 6 selected from
rac-N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide;
N-{3-[(4S,9R)-5-[(4-Fluorophenyl)methyl]-8-hydroxy-6-oxo-5-azahexacyclo[8.5.0.0^{2,15}.0^{3,12}.0^{4,9}.0^{11,13}]pentadec-7-en-7-yl]-1,1-dioxo-4H-1λ⁶,2,4-benzothiadiazin-7-yl}methanesulfonamide;
rac-N-{3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide; and
N-{3-[(1R,10S)-11-[(4-Fluorophenyl)methyl]-14-hydroxy-12-oxo-11-azahexacyclo[8.4.0.0^{2,7}.0^{3,5}.0^{4,9}.0^{6,8}]tetradec-13-en-13-yl]-1,1-dioxo-4H-1λ⁶,2,4-benzothiadiazin-7-yl}methanesulfonamide,
or a pharmaceutically acceptable salt or tautomer thereof.

8. A pharmaceutically acceptable composition comprising a compound of claim 1 or a pharmaceutically acceptable salt or tautomer thereof, and a pharmaceutically acceptable carrier.

9. A method of inhibiting hepatitis C virus replication comprising exposing hepatitis C virus to a therapeutically effective amount of a compound of claim 1.

10. The method of claim 9 wherein the inhibition of replication occurs in the presence of one or more additional therapeutic agents selected from the group consisting of an antibiotic, an antiemetic agent, an antidepressant, an antifungal agent, an anti-inflammatory agent, an antiviral agent, an anticancer agent, an immunomodulatory agent, an α-interferon, a β-interferon, a ribavirin, an alkylating agent, a hormone, a cytokine and a toll-like receptor modulator.

11. A method for treating or preventing hepatitis C virus infection in a mammal in need thereof, comprising administering to the mammal a therapeutically or prophylactically effective amount of a compound of claim 1.

12. The method of claim 11 wherein the mammal is a human.

13. The method of claim 11 further comprising administering one or more additional therapeutic agents to the mammal.

14. The method of claim 13 wherein the additional therapeutic agent is selected from the group consisting of an antibiotic, an antiemetic agent, an antidepressant, an antifungal agent, an anti-inflammatory agent, an antiviral agent, an anticancer agent, an immunomodulatory agent, an α-interferon, a β-interferon, a ribavirin, an alkylating agent, a hormone, a cytokine and a toll-like receptor modulator.

15. The method of claim 14 further comprising the additional therapeutic agent that is ITMN-191, or a pharmaceutically acceptable salt thereof.

16. The method of claim 14 further comprising the additional therapeutic agent that is R7128, or a pharmaceutically acceptable salt thereof.
